(19) United States
(12) Patent Application Publication
(10) Pub. No.: US 2022/0144663 A1
(43) Pub. Date: May 12, 2022

(54) Polyoxometalates Comprising Transition Metals

(71) Applicants: Ulrich Kortz, Bremen (DE); Hafiz M. Qasim, Bremen (DE); Ali S. Mougharbel, Bremen (DE); Bassem S. Bassil, Fanar (LB); Wassim W. Ayass, Berlin (DE)

(72) Inventors: Ulrich Kortz, Bremen (DE); Hafiz M. Qasim, Bremen (DE); Ali S. Mougharbel, Bremen (DE); Bassem S. Bassil, Fanar (LB); Wassim W. Ayass, Berlin (DE)

(21) Appl. No.: 17/430,982

(22) PCT Filed: Feb. 27, 2020
(86) PCT No.: PCT/EP2020/055103

§ 371 Date: Aug. 13, 2021

(30) Foreign Application Priority Data
Mar. 1, 2019 (EP) 19160247.3

(51) Int. Cl. C01G 41/00; B01J 23/30; B01J 37/03; C07C 315/00; B01J 38/44

(52) U.S. Cl. CPC C01G 41/006; C01P 2002/72; B01J 37/033; C07C 315/00; B01J 38/44; C01P 2002/60; C01P 2002/76; C01P 2002/77; C01P 2002/82; C01P 2002/86; C01P 2002/88; B01J 2523/69; B01J 2523/42; B01J 2523/12; B01J 2523/15; B01J 23/30

(57) ABSTRACT

The invention relates to polyoxometalates represented by the formula (Aₙ)ₘ₊{[M₆(O₂)₉][(XM′₁₀O₃₇)₃]}^{m-} or solvates thereof, corresponding supported polyoxometalates, and processes for their preparation, as well as their use in oxidative conversion of organic substrate.

### CROSS REFERENCE TO RELATED APPLICATION

The present application claims priority to and the benefit of European Application No. 19160247.3, filed on 1 Mar. 2019, which is hereby incorporated by reference in its entirety.

### FIELD OF THE INVENTION

This invention relates to new polyoxometalates (POMs). Furthermore, this invention relates to processes for the preparation of said new POMs and to their use in catalytic oxidation reactions with organic molecules.

### BACKGROUND OF THE INVENTION

POMs are a unique class of inorganic metal-oxygen clusters. They consist of a polyhedral cage structure or framework bearing a negative charge which is balanced by cations that are usually external to the cage, and may also contain internally or externally located heteroatom(s) or guest atom(s). The framework of POMs comprises a plurality of metal atoms, which can be the same or different, bonded to oxygen atoms. In the plurality of known POMs, the framework metals are dominated by a few elements including transition metals from Group 5 and Group 6 in their high oxidation states, e.g., tungsten (VI), molybdenum (VI), vanadium (V), niobium (V) and tantalum (V).

The first example in the POM family is the so-called Keggin anion [XM₁₂O₄₀]ⁿ⁻ with X being a heteroatom selected from a variety of elements, e.g., Si(IV), P(V), or Ge(IV), and M being a Group 5 or Group 6 metal such as Mo(VI) or W(VI). These anions consist of an assembly of corner- and edge-shared MO₆ octahedra of the metals of Groups 5 or 6 around a central XO₄ tetrahedron.

There have been increasing efforts towards the modification of POMs with various organic and/or transition metal complex moieties, in general, with the aim of generating new catalyst systems as well as functional materials with interesting optical, electronic, magnetic and medicinal properties. In particular, transition metal-substituted POMs (TMSPs) have attracted continuously growing attention as they can be rationally modified on the molecular level including size, shape, charge density, acidity, redox states, stability, solubility etc. In particular, the introduction of 3d, 4d, 5d and/or 4f metals in POMs is of fundamental interest en route to new, more efficient and more selective catalysts.

WO 2007/142727 A1 discloses a class of transition metal-based POMs including W having the formula [M₄(H₂O)₁₀(XW₉O₃₃)₂]^{m-} with M being a transition metal and X being selected from As, Sb, Bi, Se and Te. These POMs are particularly useful as catalysts featuring high levels of conversion in selective alkane oxidation.

WO 2008/1118619 A1 discloses another class of transition metal-based POMs including W which is illustrated by the general formula [H_{q}M₁₆X₈W₄₈O₁₈₄(HO)₃₂]^{m-} with M being selected from the group of transition metals and X being selected from As and/or P. Protocols for the preparation of these POMs were developed. Furthermore, the POMs were found to be useful catalysts.

US 2005/0112055 A1 discloses a POM including three different transition metals Ru, Zn and W with the formula Na₁₄[Ru₂Zn₂ (H₂O)₂(ZnW₉O₃₄)₂]. This particular POM was found to be highly efficient as an electrocatalyst in the generation of oxygen.

U.S. Pat. No. 4,864,041 demonstrates the general potential of transition metal-substituted POMs as catalysts for the oxidation of organic compounds in the presence of an oxygen donor. A variety of different POMs with different metal species was investigated, including those with W, Mo, V, Cu, Cr, Ni, Mn, Fe, Co, etc.

As disclosed by Bregeault et al. (J. Mol. Catal. A: Chem. 250 (2006) 177-189), POMs and peroxo-containing POMs or polyoxoperoxometalates (POPOMs) are members of the large class of oxidants and are amongst the most important for activating hydrogen peroxide. This is especially advantageous as, together with dioxygen, hydrogen peroxide is the most benign and low-waste reagent for oxidations. However, highly effective catalytic methods need to be found for its activation. In that context, transition metal polyoxoperoxo systems can be regarded as inorganic catalytic analogues of peracids. Key POM percursors or catalysts include the well-known Keggin anion [XM₁₂O₄₀]ⁿ⁻ and an example of peroxo-containing POM highly active in oxidation catalysis is Venturello ion [PW₄O₈(O₂)₈]³⁻ (J. Mol. Catal. 32 (1985) 107-110; J. Am. Chem. Soc. 117 (1995) 681-691).

Peroxo-containing molybdates and niobates, as well as lanthanum-containing peroxotungstates have been synthesized (N. Gresley et al. J. Chem. Soc., Dalton Trans. 10 (1996) 2039-2045; G-S. Kim et al., J. Med. Chem. 37 (1994) 816-820; L. Song et al., CrystEngComm. 15 (2013) 4597-4600).

Kortz and co-workers have also reported trimeric and dimeric zirconium/hafnium-peroxo-encapsulating POMs, which were able to stoichiometrically oxidize methionine to its sulfoxide form, and in the presence of peroxide further to the sulfone derivative (J. Am. Chem. Soc. 130 (2008) 6696-6697; Inorg. Chem. 49 (2010) 7-9; Chem. Eur. J. 17 (2011) 8371-8378).

However, despite these recent developments in the preparation of transition metal-containing POMs and their promising oxidation catalytic activities, there remains a need for the preparation of homogeneous and heterogeneous oxidation catalysts or catalyst systems that are highly active and selective, in particular that provide selective oxygen activation to form a selective oxygen species that can be used for selective functionalizing hydrocarbon, while being recyclable and recoverable, i.e. with reproductive efficiency and quantitative recuperation after the catalytic cycle.

Therefore, it is an object of the present invention to provide POMs containing transition metal atoms, in particular transition metal atoms. Furthermore, it is an object of the present invention to provide one or multiple processes for the preparation of said POMs. In addition, it is an object of the present invention to provide supported POMs containing transition metal atoms and optionally lanthanide metal atoms as well as one or multiple processes for the preparation of said supported POMs. Another object of the present invention is the provision of metal cluster units, in particular the provision of highly dispersed metal cluster unit particles, and processes for the preparation of said metal cluster units either in the form of a dispersion in a liquid carrier medium or in supported form, immobilized on a solid support. Finally, it is an object of the present invention to provide one or multiple processes for the homogeneous oxidative conversion of organic substrate using said optionally supported POM(s).

### SUMMARY OF THE INVENTION

An objective of the present invention among others is achieved by the provision of POMs represented by the formula

(Aₙ)^{m+}{[M₆(O₂)₉][(XM′₁₀O₃₇)₃]}^{m-}

or solvates thereof, wherein
each A independently represents a cation,
n is the number of cations,
each M is independently selected from the group consisting of Ce, Ti, Zr, Hf, Nb, and Ta,
each X is independently selected from the group consisting of Ge, Si, P, and As,
each M′ is independently selected from the group consisting of W and Mo,
m is a number representing the total positive charge m+ of n cations A and the corresponding negative charge m− of the polyanion {[M₆(O₂)₉][(XM′₁₀O₃₇)₃]}.

An objective of the present invention among others is achieved by the provision of a process for the preparation of any one of the POMs provided by the present invention, said process comprising:
(a) reacting at least one source of M, at least one source of {XM′₁₀O₃₇}, and at least one source of oxygen to form a salt of the polyanion {[M₆(O₂)₉][(XM′₁₀O₃₇)₃]} or a solvate thereof,
(b) optionally adding at least one salt of A to the reaction mixture of step (a) to form a polyoxometalate (Aₙ)^{m+}{[M₆(O₂)₉][(XM′₁₀O₃₇)₃]}^{m-} or a solvate thereof, and
(c) recovering the polyoxometalate or solvate thereof.

An objective of the present invention among others is achieved by the provision of supported POMs comprising any one of the POMs provided by the present invention or prepared according to the present invention, on a solid support.

An objective of the present invention among others is achieved by the provision of a process for the preparation of the supported POMs provided by the present invention, said process comprising the step of contacting any one of the POMs provided by the present invention or prepared according to the present invention, with a solid support.

An objective of the present invention among others is achieved by the provision of a process for the homogeneous or heterogeneous oxidative conversion of organic substrate comprising contacting said organic substrate with any one of the optionally supported POMs provided by the present invention or prepared according to the present invention.

In the context of the present invention the term transition metal comprises at least the following elements: Ti, Zr, Nb, Hf, Ta, Mo, W and Ce.

With regard to the present invention the expressions Group 1, Group 2, Group 3 etc. refer to the Periodic Table of the Elements and the expressions 3d, 4d, 4f and 5d metals refer to transition metals of respective Periods 4, 5 and 6 of the Periodic Table of the Elements, i.e., Ti is a 3d metal of Group 4, Zr is a 4d metal of Group 4, Hf is a 5d metal of Group 4, Nb is a 4d metal of Group 5, Ta is a 5d metal of Group 5, Mo is a 4d metal of Group 6, W is a 5d metal of Group 6, Ce is a 4f metal of Group 3, in the lanthanide (or lanthanoid) series (also considered as “inner transition metals”).

With regard to the present invention the term {XM′₁₀O₃₇} unit describes the structural arrangement of the XM′₁₀O₃₇ atoms in (Aₙ)^{m+}{[M₆(O₂)₉][(XM′₁₀O₃₇)₃]}^{m-}.

With regard to the present invention the term {M₆(O₂)₉} unit (or core) describes the structural arrangement of the M₆(O₂)₉ atoms in (Aₙ)^{m+}{[M₆(O₂)₉][(XM′₁₀O₃₇)₃]}^{m-}.

With regard to the present invention the term central cavity describes the space not occupied but surrounded by the M₆(O₂)₉ atoms in the {M₆(O₂)₉} unit (or core).

With regard to the present invention the term polyanion describes the negatively charged structural arrangement {[M₆(O₂)₉][(XM′₁₀O₃₇)₃]}.

With regard to the present invention the term XM′₉-based species is any precursor unit capable of forming the {XM′₁₀O₃₇} unit, which precursor unit contains 1 X atom and 9 W atoms.

With regard to the present invention the term immobilizing means to render immobile or to fix the position. In the context of a solid support the term immobilizing describes the adhesion to a surface by means of adsorption, including physisorption and chemisorption. Adsorption is based on interactions between the material to be adsorbed and the surface of the solid support such as van-der-Waals interactions, hydrogen-bonding interactions, ionic interactions, etc.

With regard to the present invention the expression primary particles of POM or POMs primary particles describes isolated particles that contain exactly one negatively charged polyanion {[M₆(O₂)₉][(XM′₁₀O₃₇)₃]}. The POMs primary particles of the present invention are substantially mono-dispersed particles, i.e. the POMs primary particles have a uniform size, corresponding to the size of one polyanion. The expression POMs secondary particles describes agglomerates of POMs primary particles.

With regard to the present invention the term supported POMs describes POMs immobilized on a solid support.

The particle size of the non-aggregated and aggregated POMs can be determined by various physical methods known in the art. If the particles are dispersed in a liquid medium, the particle size can be determined by light scattering. If the particles are supported on a solid support, solid state techniques are required for determining the particle size of the supported particles, and to distinguish between primary particles (non-aggregated) and secondary particles (aggregated). Suitable solid state techniques include scanning electron microscopy (SEM), transmission electron microscopy (TEM), powder X-ray diffraction or crystallography (powder XRD), etc. Another suitable technique for determining the particle size is pulsed chemi-/physisorption.

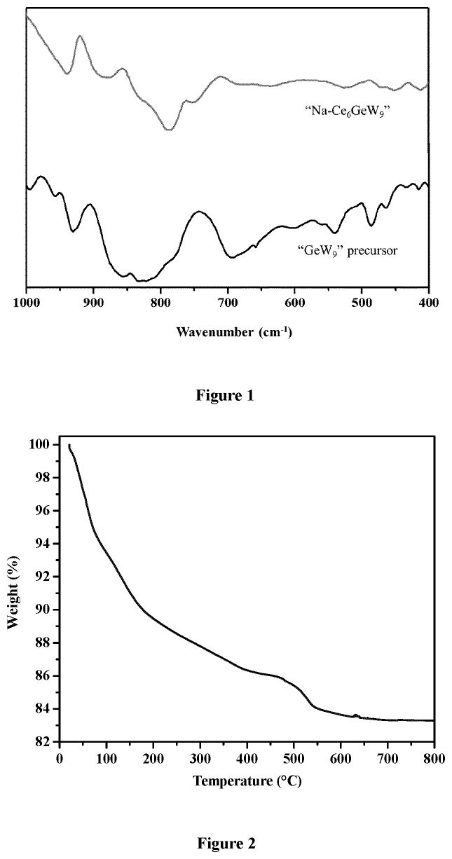

### BRIEF DESCRIPTION OF THE FIGS. 1-11

FIG. 1: Fourier Transform Infrared (FT-IR) spectrum of Na₁₀[α-GeW₉O₃₄].25H₂O (“GeW₉”) precursor (bottom curve) and Na₂₄{[Ce₆(O₂)₉][(GeW₁₀O₃₇)₃]}.100H₂O (“Na—Ce₆(GeW₁₀)₃”) (upper curve) from 1000 cm⁻¹ to 400 cm⁻¹.

FIG. 2: Thermogravimetric analysis (TGA) curve of Na₂₄{[Ce₆(O₂)₉][(GeW₁₀O₃₇)₃]}.100H₂O (“Na—Ce₆(GeW₁₀)₃”) from 0 to 800° C.

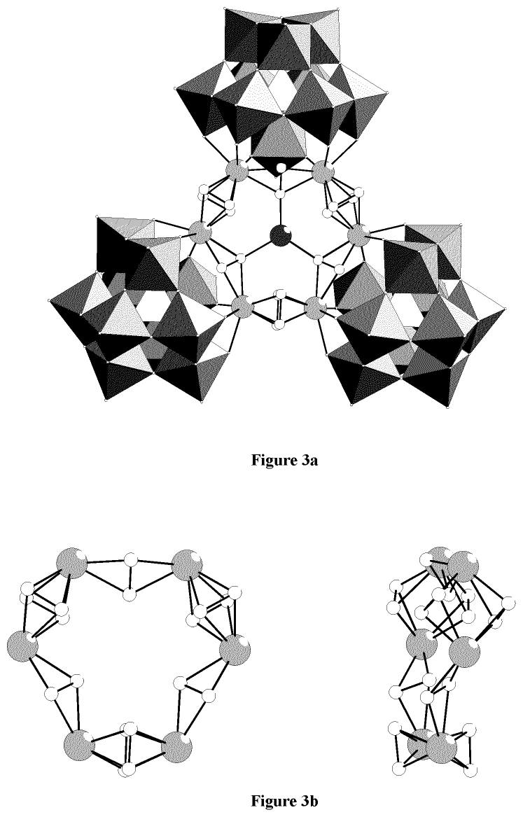

FIG. 3a: Combined polyhedral/ball-and-stick representation of the {[Ce₆(O₂)₉][(GeW₁₀O₃₇)₃]} polyanion (“Ce₆(GeW₁₀)₃”). Legend: WO₆, grey polyhedrons; GeO₄, white polyhedrons; Ce, grey balls; Na, black ball; peroxo groups, white balls.

FIG. 3b: Top and side view of the {Ce₆(O₂)₉} core. Legend: Ce, grey; peroxo groups, white.

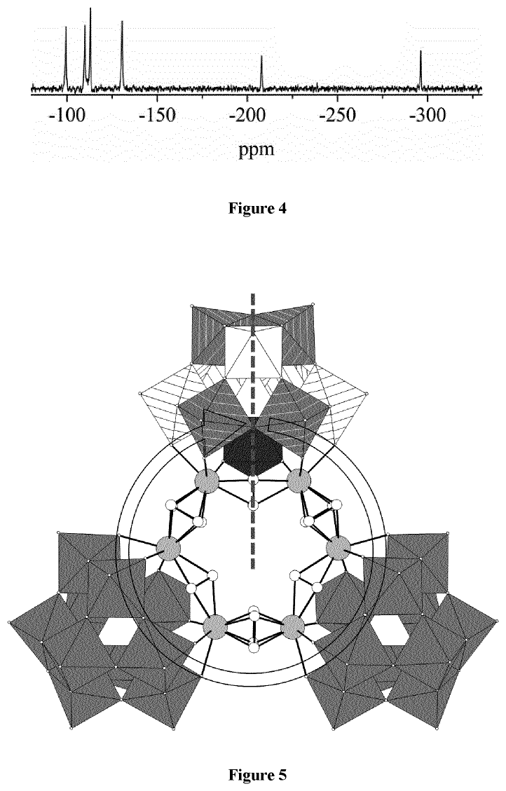

FIG. 4: ¹⁸³W NMR spectrum of Na₂₄{[Ce₆(O₂)₉][(GeW₁₀O₃₇)₃]}.100H₂O (“Na—Ce₆(GeW₁₀)₃”) recorded in H₂O/D₂O.

FIG. 5: Combined polyhedral/ball-and-stick representation of the “Ce₆(GeW₁₀)₃” polyanion showing the six types of non-equivalent W atoms.

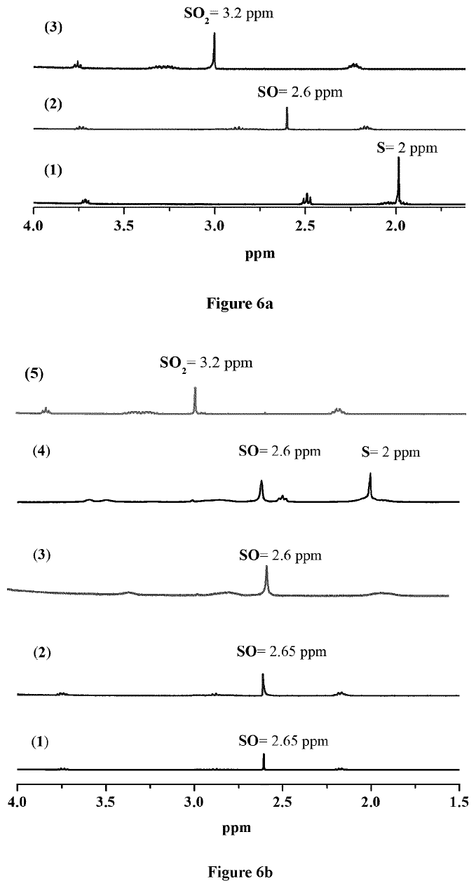

FIG. 6a: (1)¹H NMR spectra of pure DL-methionine (S) substrate; (2)¹H NMR spectra of pure methionine sulfoxide (SO; (3)¹H NMR spectra of pure methionine (SO₂).

FIG. 6b: Formation of methionine sulfoxide (SO) and methionine sulfone (SO₂) products, as measured by ¹H NMR spectra.

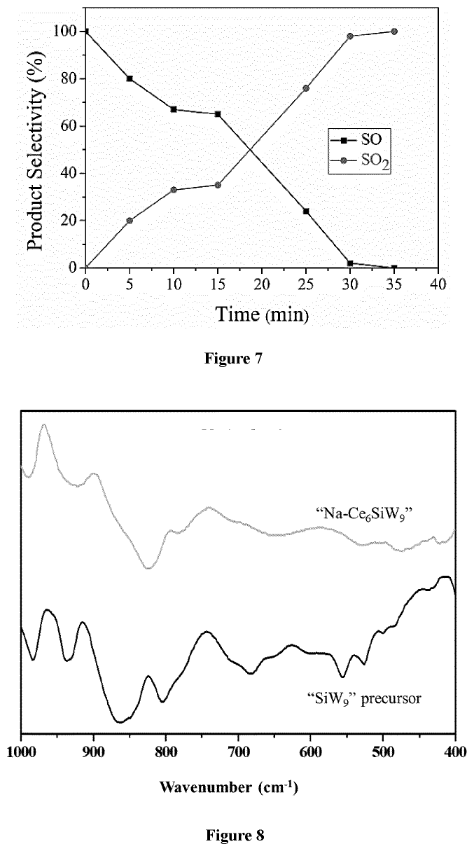

FIG. 7: Product selectivity profile, as measured by ¹H NMR.

FIG. 8: Fourier Transform Infrared (FT-IR) spectrum of Na₁₀[α-SiW₉O₃₄].25H₂O (“SiW₉”) precursor (bottom curve) and Na₂₄{[Ce₆(O₂)₉][(SiW₁₀O₃₇)₃]}.100H₂O (“Na—Ce₆(SiW₁₀)₃”) (upper curve) from 1000 cm⁻¹ to 400 cm⁻¹.

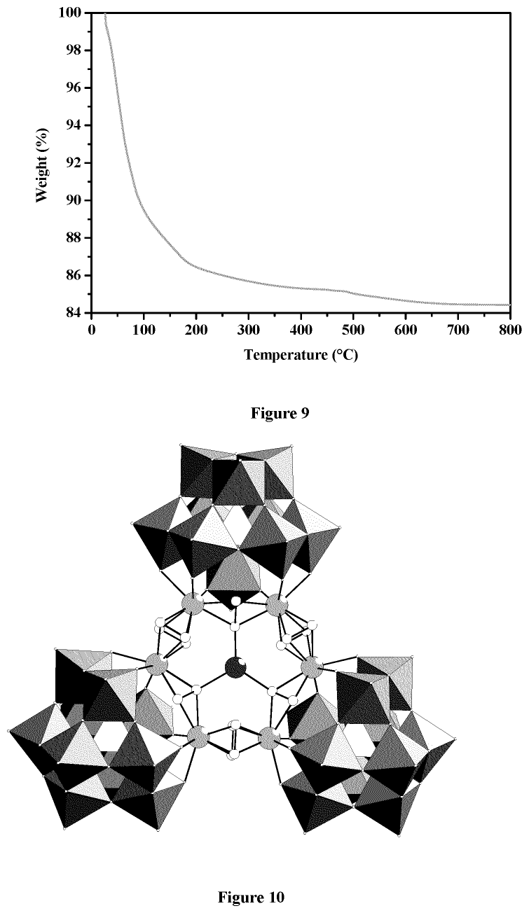

FIG. 9: Thermogravimetric analysis (TGA) curve of Na₂₄{[Ce₆(O₂)₉][(SiW₁₀O₃₇)₃]}.100H₂O (“Na—Ce₆(SiW₁₀)₃”) from 0 to 800° C.

FIG. 10: Combined polyhedral/ball-and-stick representation of the {[Ce₆(O₂)₉][(SiW₁₀O₃₇)₃]} polyanion (“Ce₆(SiW₁₀)₃”). Legend: WO₆, grey polyhedrons; SiO₄, white polyhedrons; Ce, grey balls; Na, black ball; peroxo groups, white balls.

### DETAILED DESCRIPTION

According to one embodiment, the POMs of the present invention are represented by the formula

(Aₙ)^{m+}{[M₆(O₂)₉][(XM′₁₀O₃₇)₃]}^{m-}

or solvates thereof, wherein
each A independently represents a cation, preferably each A is independently selected from the group consisting of Li, Na, K, Cs, ammonium (NH₄), tetraalkyl ammonium (NR₄), tetraalkyl phosphonium (PR₄) and combinations thereof, preferably from Li, K, Na, ammonium, tetraalkyl ammonium, tetraalkyl phosphonium and combinations thereof, more preferably wherein all A are the same, e.g. wherein all A are Na, Li or K, n is the number of cations,
each M is independently selected from the group consisting of Ce, Ti, Zr, Hf, Nb, and Ta, in particular wherein all M are the same, more particularly wherein all M are Ce, Zr or Hf, most particularly wherein all M are Ce,
each X is independently selected from the group consisting of Ge, Si, P, and As, preferably Gee^{IV}, Si^{IV}, P^{V}, and As^{V}, in particular wherein all X are the same, more particularly wherein all M are Ge^{IV} or Si^{IV},
each M′ is independently selected from the group consisting of W and Mo, particularly W,
m is a number representing the total positive charge m+ of n cations A and the corresponding negative charge m− of the polyanion {[M₆(O₂)₉][(XM′₁₀O₃₇)₃]}.

The polyanion {[M₆(O₂)₉][(XM′₁₀O₃₇)₃]} of the POM according to the invention has been found to comprise a cyclic assembly of three {XM′₁₀O₃₇} units encapsulating the {M₆(O₂)₉} unit or core (FIGS. 3a, 5 and 10). The central {M₆(O₂)₉} core, in the form of an M-peroxo wheel, is composed of six equivalent M atoms in the +4 (e.g. is M is Ce, Ti, Zr or Hf) or +5 (e.g. if M is Nb or Ta) oxidation stage. Two M atoms occupy the lacunary site of each {XM′₁₀O₃₇} unit, and are further linked via a side-on μ-peroxo bridge (M-(O₂)-M). In turn, the three monomers containing said {XM′₁₀O₃₇} unit, two M atoms and μ-peroxo bridge, are connected via two side-on μ-peroxo bridges (M-(O₂)₂-M), one above and one below the overall plane of the molecule (i.e. di-bridges), giving the molecule a C₃ᵥ point group symmetry. The M-oxo bond lengths range from 2.237(15) to 2.446(16) Å, and that of the M-peroxo range from 2.307(15) to 2.446(14) Å. The O—O bond lengths are between 1.489(19) and 1.52(2) Å, close to the reported value of 1.49 Å for the crystal structure of hydrogen peroxide as disclosed in Abrahans et al. (ActaCryst. 4 (1951) 15-20).

The structure of the {M₆(O₂)₉}⁶⁺ core in the POM according to the invention is very close to that of {Zr₆(O₂)₆(OH)₆}⁶⁺ or {Hf₆(O₂)₆(OH)₆}⁶⁺ in respectively {Zr₆(O₂)₆(OH)₆(SiW₁₀O₃₆)₃}¹⁸⁻ or {Hf₆(O₂)₆(OH)₆(SiW₁₀O₃₆)₃}¹⁸⁻ as disclosed in Bassil et al. (J. Am. Chem. Soc. 130 (2008) 6696-6697), with one fundamental difference: each M atom in the present polyanion is coordinated to three side-on peroxo bridges and three oxo ligands from the lacunary {XM′₁₀O₃₇} units, making them each nonacoordinated with O, while the Zr/Hf centers in {Zr₆(O₂)₆(OH)₆(SiW₁₀O₃₆)₃}¹⁸⁻ or {Hf₆(O₂)₆(OH)₆(SiW₁₀O₃₆)₃}¹⁸⁻ are each coordinated to two side-on peroxo bridges, two hydroxo bridges, and two oxo ligands from the lacunary {SiW₁₀O₃₆} unit, them each octacoordinated with O. It is this difference in coordination number that allows the polyanion {[M₆(O₂)₉][(XM′₁₀O₃₇)₃]} of the POM according to the invention to encapsulate nine peroxo groups versus maximum six peroxo groups in the POMs of Bassil et al.

As detailed above, in the present POMs, the peroxo groups show two different sets of connectivities in the structure, the first one bridging the two M atoms that are connected to a {XM′₁₀O₃₇} unit forming a so-called “monomer” (M-(O₂)-M) and the second one bridging two M atoms of two different “monomers” (M-(O₂)₂-M). Without wishing to be bound by any theory, it is believed that the three peroxo groups found in the (M-(O₂)-M) bridges can be released, such as by heating, and made available for instance for selective functionalizing, and in particular oxidation, of substrate molecules. As a result, if M is Ce, the oxidation state of the corresponding Ce atoms is reduced from 4+ to 3+. If M is Ti, Zr, Hf, Nb or Ta, there is no change in the oxidation state of the corresponding M atoms. Also, without wishing to be bound by any theory, it is believed that the six peroxo groups found in the (M-(O₂)₂-M) di-bridges can release six oxygens to substrate molecules resulting in the formation of μ-oxo/hydroxo bridges in the POM structure.

In a preferred variant of the first embodiment, all M are the same, all M′ are the same, and all X are the same.

In a second preferred variant of the first embodiments or of the preferred variant of said embodiments, M atoms are independently selected from Ce, Zr and Hf, preferably all M are the same, more preferably M atoms are Ce.

In a third preferred variant of the first embodiments or of the first or second preferred variant of said embodiments, the {M₆(O₂)₉} unit has a central cavity.

In a preferred embodiment, in the {XM′₁₀O₃₇} unit all of the 37 O have an oxidation state of −2, all of the 10 M′ have an oxidation state of +6 and heteroatom X has an oxidation state of +4 or +5, in particular X is selected from the group consisting of Gem^{IV}, Si^{IV}, P^{V}, and As^{V}, more particularly from Ge^{IV} and Si^{IV} and M′ is selected from the group consisting of W^{VI} and Mo^{VI}, more particularly W^{VI}.

In a further preferred embodiment, all M are the same and all M atoms are selected from the group consisting of Ce^{IV}, Ti^{IV}, Zr^{IV}, Hf^{IV}, Nb^{V}, and Ta^{V}, in particular all M atoms are Ce^{IV}, Zr^{IV} or Hf^{IV}, more particularly Ce^{IV}.

In the POMs of the present invention, the cation A is independently selected from the group consisting of Li, Na, K, Cs, ammonium (NH₄), tetraalkyl ammonium (NR₄), tetraalkyl phosphonium (PR₄) and combinations thereof, preferably from Li, K, Na, ammonium, tetraalkyl ammonium and combinations thereof, more preferably wherein all A are the same, e.g. all A are Na, Li or K. Suitable examples of tetraalkyl ammonium groups are NR₄ wherein R is independently selected from C₁-C₁₂ alkyl or aryl groups, in particular from C₂-C₄ alkyl groups or from phenyl groups; most often, all R are the same, specific examples being tetraethylammononium, tetra-n-propylammonium, tetra-i-propylammonium, tetra-n-butylammonium (TBA), tetra-i-butylammonium, tetra-sec-butylammonium, and tetra-t-butylammonium, in particular TBA. Suitable examples of tetraalkyl phosphonium groups are PR₄ wherein R is independently selected from C₁-C₁₂ alkyl or aryl groups, in particular from C₂-C₄ alkyl groups or from phenyl groups; most often, all R are the same, specific examples being tetraethylphosphonium, tetra-n-propylphosphonium, tetra-i-propylphosphonium, tetra-n-butylphosphonium (TBA), tetra-i-butylphosphonium, tetra-sec-butylammonium, and tetra-t-butylphosphonium.

The number n of cations is dependent on the nature of cation(s) A, namely its/their valence, and the negative charge m of the polyanion which has to be balanced. In any case, the overall charge of all cations A is equal to the charge of the polyanion. In turn, the charge m of the polyanion is dependent on the nature and oxidation state of the metals M and M′, the nature and oxidation state of the heteroatoms X and the number of peroxo groups q, hydroxo groups p and oxo groups r. Thus, m depends on the oxidation state of the atoms present in the polyanion, e.g., it follows from the oxidation states of O (−2), H (+1), X (preferably +4 for Ge^{IV} or Si^{IV} or +5 for As^{V} or P^{V}), M (preferably +4 for Ce^{IV}, Ti^{IV}, Zr^{IV} or Hf^{IV} or +5 for Nb^{V} or Ta^{V}), and M′ (usually +6). In some embodiments, m ranges from 20 to 28, preferably from 21 to 26. In particular, m is 21 or 24. In a preferred embodiment, m is 24. Thus, n can generally range from 20 to 28, preferably from 21 to 26. In particular, n is 21 or 24. In a preferred embodiment, n is 24.

Generally, A is acting as counterion of the POM and is positioned outside of the polyanion. However, it is also possible that some of the cations A are located within the polyanion. As the {M₆(O₂)₉} unit has a central cavity, it is also possible that some of the cations A are located within the central cavity. Without being bound by any theory, it is believed that cation(s) A located within the central cavity may stabilize the cyclic structure.

Thus, in a preferred embodiment, the invention relates to a POM (Aₙ)^{m+}{[M₆(O₂)₉][(XM′₁₀O₃₇)₃]}^{m-}, wherein M is Ce, Zr or Hf, preferably Ce.

Thus, in a preferred embodiment, the invention relates to a POM (Aₙ)^{m+}{[M₆(O₂)₉][(XM′₁₀O₃₇)₃]}^{m-}, wherein X is Ge or Si.

Thus, in a preferred embodiment, the invention relates to a POM (Aₙ)^{m+}{[M₆(O₂)₉][(XM′₁₀O₃₇)₃]}^{m-}, wherein M′ is W or Mo, preferably W.

Thus, in a preferred embodiment, the invention relates to a POM (Aₙ)^{m+}{[M₆(O₂)₉][(XM′₁₀O₃₇)₃]}^{m-}, wherein M is Ce, X is Ge or Si, and M′ is W.

Suitable examples of POMs according to the invention are represented by the formulae

(Aₙ)^{m+}{[M₆(O₂)₉][(XM′₁₀O₃₇)₃]}^{m-},such as

(Aₙ)^{m+}{[M₆(O₂)₉][(GeW₁₀O₃₇)₃]}^{m-},

(Aₙ)^{m+}{[M₆(O₂)₉][(GEMo₁₀O₃₇)₃]}^{m-},

(Aₙ)^{m+}{[M₆(O₂)₉][(SiW₁₀O₃₇)₃]}^{m-},

(Aₙ)^{m+}{[M₆(O₂)₉][(SiMo₁₀O₃₇)₃]}^{m-},

(Aₙ)^{m+}{[M₆(O₂)₉][(AsW₁₀O₃₇)₃]}^{m-},

(Aₙ)^{m+}{[M₆(O₂)₉][(AsMo₁₀O₃₇)₃]}^{m-},

(Aₙ)^{m+}{[M₆(O₂)₉][(PW₁₀O₃₇)₃]}^{m-},

(Aₙ)^{m+}{[M₆(O₂)₉][(PMo₁₀O₃₇)₃]}^{m-},

for instance

(Aₙ)²⁴⁺{[Ce₆(O₂)₉][(GeW₁₀O₃₇)₃]}²⁴⁻,

(Aₙ)²⁴⁺{[Ce₆(O₂)₉][(GeMo₁₀O₃₇)₃]}²⁴⁻,

(Aₙ)²⁴⁺{[Ce₆(O₂)₉][(SiW₁₀O₃₇)₃]}²⁴⁻,

(Aₙ)²⁴⁺{[Ce₆(O₂)₉][(SiMo₁₀O₃₇)₃]}²⁴⁻,

(Aₙ)²¹⁺{[Ce₆(O₂)₉][(AsW₁₀O₃₇)₃]}²¹⁻,

(Aₙ)²¹⁺{[Ce₆(O₂)₉][(AsMo₁₀O₃₇)₃]}²¹⁻,

(Aₙ)²¹⁺{[Ce₆(O₂)₉][(PW₁₀O₃₇)₃]}²¹⁻,

(Aₙ)²¹⁺{[Ce₆(O₂)₉][(PMo₁₀O₃₇)₃]}²¹⁻,

(Aₙ)²⁴⁺{[Zr₆(O₂)₉][(GeW₁₀O₃₇)₃]}²⁴⁻,

(Aₙ)²⁴⁺{[Zr₆(O₂)₉][(GeMo₁₀O₃₇)₃]}²⁴⁻,

(Aₙ)²⁴⁺{[Zr₆(O₂)₉][(SiW₁₀O₃₇)₃]}²⁴⁻,

(Aₙ)²⁴⁺{[Zr₆(O₂)₉][(SiMo₁₀O₃₇)₃]}²⁴⁻,

(Aₙ)²¹⁺{[Zr₆(O₂)₉][(AsW₁₀O₃₇)₃]}²¹⁻,

(Aₙ)²¹⁺{[Zr₆(O₂)₉][(AsMo₁₀O₃₇)₃]}²¹⁻,

(Aₙ)²¹⁺{[Zr₆(O₂)₉][(PW₁₀O₃₇)₃]}²¹⁻,

(Aₙ)²¹⁺{[Zr₆(O₂)₉][(PMo₁₀O₃₇)₃]}²¹⁻,

(Aₙ)²⁴⁺{[Hf₆(O₂)₉][(GeW₁₀O₃₇)₃]}²⁴⁻,

(Aₙ)²⁴⁺{[Hf₆(O₂)₉][(GeMo₁₀O₃₇)₃]}²⁴⁻,

(Aₙ)²⁴⁺{[Hf₆(O₂)₉][(SiW₁₀O₃₇)₃]}²⁴⁻,

(Aₙ)²⁴⁺{[Hf₆(O₂)₉][(SiMo₁₀O₃₇)₃]}²⁴⁻,

(Aₙ)²¹⁺{[Hf₆(O₂)₉][(AsW₁₀O₃₇)₃]}²¹⁻,

(Aₙ)²¹⁺{[Hf₆(O₂)₉][(AsMo₁₀O₃₇)₃]}²¹⁻,

(Aₙ)²¹⁺{[Hf₆(O₂)₉][(PW₁₀O₃₇)₃]}²¹⁻,

(Aₙ)²¹⁺{[Hf₆(O₂)₉][(PMo₁₀O₃₇)₃]}²¹⁻.

The invention also includes solvates of the present POMs. A solvate is an association of solvent molecules with a POM. Preferably, water is associated with the POMs and thus, the POMs according to the invention can in particular be represented by the formulae

(Aₙ)^{m+}{[M₆(O₂)₉][(XM′₁₀O₃₇)₃]}^{m-}.wH₂O,

wherein A, n, m, M, M′ and X are the same as defined above, and w represents the number of attracted water molecules per polyanion {[M₆(O₂)₉][(XM′₁₀O₃₇)₃]} and mostly depends on the type of cations A. In some embodiments w is an integer that ranges from 1 to 200, preferably from 50 to 150, more preferably from 70 to 130, most preferably from 80 to 120, for instance from 90 to 110, such as about 100.

Generally, the w H₂O molecules are positioned outside of the polyanion. However, it is also possible that some of the w H₂O molecules are located within the polyanion. In case the {M₆(O₂)₉} unit has a central cavity, it is also possible that some of the w H₂O molecules are located within the central cavity.

Suitable examples of the POM solvates according to the invention are represented by the formulae

(Aₙ)^{m+}{[M₆(O₂)₉ᵣ][(XM′₁₀O₃₇)₃]}^{m-}.wH₂O, such as

(Aₙ)^{m+}{[M₆(O₂)₉][(GeW₁₀O₃₇)₃]}^{m-}.wH₂O,

(Aₙ)^{m+}{[M₆(O₂)₉][(GeMo₁₀O₃₇)₃]}^{m-}.wH₂O,

(Aₙ)^{m+}{[M₆(O₂)₉][(SiW₁₀O₃₇)₃]}^{m-}.wH₂O,

(Aₙ)^{m+}{[M₆(O₂)₉][(SiMo₁₀O₃₇)₃]}^{m-}.wH₂O,

(Aₙ)^{m+}{[M₆(O₂)₉][(AsW₁₀O₃₇)₃]}^{m-}.wH₂O,

(Aₙ)^{m+}{[M₆(O₂)₉][(AsMo₁₀O₃₇)₃]}^{m-}.wH₂O,

(Aₙ)^{m+}{[M₆(O₂)₉][(PW₁₀O₃₇)₃]}^{m-}.wH₂O,

(Aₙ)^{m+}{[M₆(O₂)₉][(PMo₁₀O₃₇)₃]}^{m-}.wH₂O,

for instance

(Aₙ)²⁴⁺{[Ce₆(O₂)₉][(GeW₁₀O₃₇)₃]}²⁴⁻.wH₂O,

(Aₙ)²⁴⁺{[Ce₆(O₂)₉][(GeMo₁₀O₃₇)₃]}²⁴⁻.wH₂O,

(Aₙ)²⁴⁺{[Ce₆(O₂)₉][(SiW₁₀O₃₇)₃]}²⁴⁻.wH₂O,

(Aₙ)²⁴⁺{[Ce₆(O₂)₉][(SiMo₁₀O₃₇)₃]}²⁴⁻.wH₂O,

(Aₙ)²¹⁺{[Ce₆(O₂)₉][(AsW₁₀O₃₇)₃]}²¹⁻.wH₂O,

(Aₙ)²¹⁺{[Ce₆(O₂)₉][(AsMo₁₀O₃₇)₃]}²¹⁻.wH₂O,

(Aₙ)²¹⁺{[Ce₆(O₂)₉][(PW₁₀O₃₇)₃]}²¹⁻.wH₂O,

(Aₙ)²¹⁺{[Ce₆(O₂)₉][(PMo₁₀O₃₇)₃]}²¹⁻.wH₂O,

(Aₙ)²⁴⁺{[Zr₆(O₂)₉][(GeW₁₀O₃₇)₃]}²⁴⁻.wH₂O,

(Aₙ)²⁴⁺{[Zr₆(O₂)₉][(GeMo₁₀O₃₇)₃]}²⁴⁻.wH₂O,

(Aₙ)²⁴⁺{[Zr₆(O₂)₉][(SiW₁₀O₃₇)₃]}²⁴⁻.wH₂O,

(Aₙ)²⁴⁺{[Zr₆(O₂)₉][(SiMo₁₀O₃₇)₃]}²⁴⁻.wH₂O,

(Aₙ)²¹⁺{[Zr₆(O₂)₉][(AsW₁₀O₃₇)₃]}²¹⁻.wH₂O,

(Aₙ)²¹⁺{[Zr₆(O₂)₉][(AsMo₁₀O₃₇)₃]}²¹⁻.wH₂O,

(Aₙ)²¹⁺{[Zr₆(O₂)₉][(PW₁₀O₃₇)₃]}²¹⁻.wH₂O,

(Aₙ)²¹⁺{[Zr₆(O₂)₉][(PMo₁₀O₃₇)₃]}²¹⁻.wH₂O,

(Aₙ)²⁴⁺{[Hf₆(O₂)₉][(GeW₁₀O₃₇)₃]}²⁴⁻.wH₂O,

(Aₙ)²⁴⁺{[Hf₆(O₂)₉][(GeMo₁₀O₃₇)₃]}²⁴⁻.wH₂O,

(Aₙ)²⁴⁺{[Hf₆(O₂)₉][(SiW₁₀O₃₇)₃]}²⁴⁻.wH₂O,

(Aₙ)²⁴⁺{[Hf₆(O₂)₉][(SiMo₁₀O₃₇)₃]}²⁴⁻.wH₂O,

(Aₙ)²¹⁺{[Hf₆(O₂)₉][(AsW₁₀O₃₇)₃]}²¹⁻.wH₂O,

(Aₙ)²¹⁺{[Hf₆(O₂)₉][(AsMo₁₀O₃₇)₃]}²¹⁻.wH₂O,

(Aₙ)²¹⁺{[Hf₆(O₂)₉][(PW₁₀O₃₇)₃]}²¹⁻.wH₂O,

(Aₙ)²¹⁺{[Hf₆(O₂)₉][(PMo₁₀O₃₇)₃]}²¹⁻.wH₂O.

In a preferred embodiment, water molecules, if present at all, can coordinate A cations. In a preferred embodiment, the water molecules are not directly attached to the POM framework (Aₙ)^{m+}{[M₆(O₂)₉[(XM′₁₀O₃₇)₃]}^{m-} by coordination but rather indirectly by hydrogen-bonding as water of crystallization. Thus, in a preferred embodiment, the attracted water molecules, if present at all, are coordinated to A cations and/or possibly exhibit weak interactions by hydrogen bonding and/or the attracted water molecules, if present at all, are lattice water molecules.

Specific examples of structures of specific POMs of the present invention are also illustrated in FIGS. 3, 5 and 10.

The present POMs are especially advantageous in that they can easily be prepared in a one-pot reaction from Keggin-based POM precursors and in that the polyanions are stable in solution or can be immobilized on a solid support, therefore forming supported POMs. Also, the present POMs show a unique combination of (i) exceptionally high catalytic activity and the (ii) ability of being regenerated and recovered very efficiently maintaining most, if not all, of their catalytic activity. In comparison to most known transition metal-substituted POMs (TMSPs), the present POMs are especially advantageous in that they act as a peroxo unit carrier for oxygen transfer, which can be unloaded and reloaded, and leads to highly selective catalytic oxidation with a source of oxygen, in particular with a source of peroxo groups, e.g. with hydrogen peroxide activation. In particular, the POMs of the present invention encapsulate nine peroxo groups that can release oxygen species to be used for selective functionalizing, and in particular oxidation, of hydrocarbons.

The invention is further directed to a process for preparing POMs according to the invention.

A process for preparing POMs according to the present invention comprises:
(a) reacting at least one source of M, at least one source of {XM′₁₀O₃₇}, and a source of oxygen to form a salt of the polyanion {[M₆(O₂)₉][(XM′₁₀O₃₇)₃]} or a solvate thereof,
(b) optionally adding at least one salt of A to the reaction mixture of step (a) to form a polyoxometalate (Aₙ)^{m+}{[M₆(O₂)₉][(XM′₁₀O₃₇)₃]}^{m-} or a solvate thereof, and
(c) recovering the polyoxometalate or solvate thereof.
   wherein A, n, m, M, M′ and X are the same as defined above.

In step (a) of said process at least one source of oxygen is used, especially one source of oxygen, in particular at least one source of peroxo groups (O₂²⁻), more particularly one source of peroxo groups. Generally, in a preferred embodiment of the present invention, the at least one source of oxygen can be a gas containing dioxygen (O₂), e.g. air, ozone (O₃), nitrous oxide (N₂O), peroxodisulfate, or a peroxide such as hydrogen peroxide (H₂O₂), an organic hydroperoxide (ROOH) e.g. tert-butylhydroperoxide, an organic dialkylperoxide (ROOR) e.g. tert-butyl peroxide, an organic diacyl peroxide (RC(O)OOC(O)R) e.g. diacetyl peroxide, a peroxy acid (RC(O)OOH) e.g. peracetic acid or m-chloroperbenzoic acid, or a peroxy ester (RC(O)OOR′). In an especially preferred embodiment, the source of oxygen is air, N₂O or a peroxide, in particular hydrogen peroxide. Hydrogen peroxide is especially advantageous as it is the most benign and low-waste reagent.

In step (a) of said process at least one source of M is used, especially one source of M. Generally, in a preferred embodiment of the present invention, for M comprising Ce, the at least one source of M can be a water-soluble salt of Ce, such CeCl₃, Ce(NO₃)₃, Ce₂(SO₄)₃, or Ce(CH₃COO)₃; for M comprising Ti, the at least one source M can be a water-soluble salt of Ti, titanium oxide such as TiO₂ or a water-soluble salt of Ti, such as TiCl₄; for M comprising Zr, the at least one source of M can be a water-soluble salt of Zr, such as ZrCl₄, Zr(NO₃)₄, Zr(SO₄)₂, or Zr(CH₃COO)₄; for M comprising Hf, the at least one source of M can be a water-soluble salt of Hf, such as such as HfCl₄, Hf(NO₃)₄, Hf(SO₄)₂, or Hf(CH₃COO)₄; for M comprising Nb, the at least one source of M can be niobium oxide such as Nb₂O₅ or a water-soluble salt of Nb, such as NbCl₃, NbCl₅, or Nb₂(SO₄)₃; for M comprising Ta, the at least one source of M can be tantalum oxide such as Ta₂O₅ or a water-soluble salt of Ta, such as TaCl₃ or TaCl₅.

In step (a) of said process at least one source of {XM′₁₀O₃₇} is used, especially one source of {XM′₁₀O₃₇}. Generally, in a preferred embodiment of the present invention, the at least one source of {XM′₁₀O₃₇} is an XM′₉-based species, preferably an XM′₉O₃₄-based species, more preferably a water soluble XM′₉O₃₄-based species such as a water soluble [XM′₉O₃₄]¹⁰⁻ salt, for instance a [XM′₉O₃₄]¹⁰⁻ salt of lithium, sodium, potassium, hydrogen or a combination thereof, more preferably a [XM′₉O₃₄]¹⁰⁻ salt of lithium, sodium, potassium, or a combination thereof. In a preferred embodiment, the at least one source of {XM′₁₀O₃₇} is Na₁₀[SiW₉O₃₄] and/or Na₁₀[GeW₉O₃₄], for instance Na₁₀[SiW₉O₃₄] and/or Na₁₀[GeW₉O₃₄] as prepared according to Herve et al. (Inorg. Chem., Vol. 16, No. 8, 2115-2117, 1977), incorporated herewith in its entirety.

Generally, in a preferred embodiment of the present invention, at least one salt of A is used in step (a), added as a solid or in the form of an aqueous solution. The counterions of A can be selected from the group consisting of any stable, non-reducing, water-soluble anion, e.g., halides, nitrate, sulfate, acetate, phosphate. Preferably, halides, nitrate and sulfate salts are used, for instance Na₂SO₄, Li₂SO₄, K₂SO₄, NaNO3, LiNO₃, KNO₃, NaCl, LiCl, KCl, NaBr, KBr or NaI, in particular NaCl, LiCi, KCl, NaBr, KBr or NaI, more preferably in the form of an aqueous solution.

Furthermore, in a preferred embodiment of the present invention, in step (a) of said process, the concentration of the metal ions M originating from the at least one source of M ranges from 0.001 to 1 mole/l, preferably from 0.002 to 0.5 mole/l, more preferably from 0.005 to 0.1 mole/l, and the concentration of the heteroatoms X originating from the sources of {XM′₁₀O₃₇} ranges from 0.0005 to 0.05 mole/l, preferably 0.001 to 0.01 mole/l. In a further preferred embodiment of the present invention, in step (a) of said process, the molar ratio M:X ranges from 1:1 to 3:1, for instance from 1.5:1 to 2.5:1 or from 1.8:1 to 2.2:1.

Furthermore, in a preferred embodiment of the present invention, in step (a) of said process, if the source of oxygen is hydrogen peroxide, it is most often added in the form of aqueous hydrogen peroxide, generally at a concentration of 3 to 90 wt % in water, preferably 10 to 50 wt %, such as about 30 wt % in water.

Furthermore, in a preferred embodiment of the present invention, in step (a) of said process, if the source of oxygen is O₂ such as air, O₃ or N₂O, it is added in excess of M. If the source of oxygen is peroxodisulfate or a peroxide, e.g. hydrogen peroxide, it may be added in a molar ratio of peroxide to M of at least 3:1, in particular of 3:1 to 10:1, more particularly of 3.5 to 7, for instance of 4 to 5.

In a preferred embodiment, step (a) of said process is carried out in an aqueous solution. In a preferred embodiment, minor amounts of organic solvent, such as, 40 to 0.01 vol % based on the total volume of the reaction mixture, preferably 30 to 0.05 vol %, 20 to 0.1 vol %, 10 to 0.2 vol %, 5 to 0.5 vol % or 3 to 1 vol %, may be added to the aqueous solution. In particular, if any of the starting materials has only a low solubility in water it is possible to dissolve the respective starting material in a small volume of organic solvent and then adding this solution to an aqueous solution of the remaining starting materials or vice versa. Examples of suitable organic solvents include, but are not limited to acetonitrile, acetone, toluene, DMF, DMSO, ethanol, methanol, n-butanol, sec-butanol, isobutanol and mixtures thereof. It is also possible to use emulsifying agents to allow the reagents of step (a) of said process to undergo a reaction.

Furthermore, in a preferred embodiment, the pH of the aqueous solution in step (a) of said process ranges from 1 to 10, preferably from 1.5 to 9 and more preferably from 2 to 8. Most preferably, the pH is from about 3 to about 7, for instance from about 3.5 to about 6.5.

Generally, in an embodiment of the present invention, additional base or acid solution can be used for adjusting the pH to a certain value. Suitable examples include aqueous sodium hydroxide or H₂SO₄ solution having a concentration of from 0.1 to 12 M, preferably 0.1 to 8 M, more preferably from 0.1 to 6 M, for instance about 0.1 M or about 1 M.

In the context of the present invention the pH of the aqueous solution in step (a) of said process refers to the pH as measured at the beginning of the reaction, before potential heating. pH values are at 20° C., and are determined to an accuracy of ±0.05 in accordance with the IUPAC Recommendations 2002 (RP. Buck et al., Pure Appl. Chem., Vol. 74, No. 11, pp. 2169-2200, 2002). A suitable and commercially available instrument for pH measurement is the Mettler Toledo FE20 pH meter. The pH calibration is carried out as 2-point calibration using a pH=4.01 standard buffer solution and a pH=7.00 standard buffer solution. The resolutions are: 0.01 pH; 1 mV; and 0.1° C. The limits of error are: ±0.01 pH; ±1 mV; and ±0.5° C.

Any of the above additives may be used individually or in combination as well as in combination with other additives commonly used in the art.

In step (a) of the process of the present invention, the reaction mixture is typically heated to a temperature of from 20° C. to 100° C., preferably from 40° C. to 80° C., preferably from 45° C. to 60° C., such as about 50° C.

In step (a) of the process of the present invention, the reaction mixture is typically heated for about 10 min to about 4 h, more preferably for about 30 min to 2 h, most preferably for about 90 min. Further, it is preferred that the reaction mixture is stirred during step (a).

With regard to the present invention the term crude mixture relates to an unpurified mixture after a reaction step and is thereby used synonymously with reaction mixture of the preceding reaction step.

In a preferred embodiment of the process of the present invention, between step (a) and (b), the crude mixture is filtered. Preferably, the crude mixture is filtered immediately after the end of step (a), i.e. immediately after the stirring is turned off, and is then optionally cooled. Alternatively, if applicable the heated crude mixture is cooled first, preferably to room temperature, and subsequently filtered. The purpose of this filtration is to remove solid impurities after step (a). Thus, the product of step (a) remains in the filtrate.

In a preferred embodiment, in case cation A is not present in the crude mixture or filtrate already, or the concentration of A in the crude mixture or filtrate should be increased, in step (b) of the process, a salt of the cation A can be added to the reaction mixture of step (a) of the process or to its corresponding filtrates to form (Aₙ)^{M+}{[M₆(O₂)₉][(XM′₁₀O₃₇)₃]}^{m-}. Preferably, the salt of A is added as a solid or in the form of an aqueous solution. The counterions of A can be selected from the group consisting of any stable, non-reducing, water-soluble anion, e.g., halides, nitrate, sulfate, acetate, phosphate. Preferably, the acetate or phosphate salt is used. However, the addition of extra cations A in step (b) of the process is not necessary if the desired cations are already present during step (a) of the process, for example, as a component of any of the sources of M or {XM′₁₀O₃₇}. Preferably, all desired cations are already present during step (a) of the process, so that optional addition of extra cations is not necessary.

In step (c) of the process of the present invention, the POMs according to the invention or solvates thereof, formed in step (a) or (b) of said process, are recovered. For example, isolation of the POMs or solvates thereof can be effected by common techniques including bulk precipitation or crystallization. In a preferred embodiment of the present invention the POMs are isolated as crystalline or amorphous solids, preferably as crystalline solids. Crystallization or precipitation can be effected by common techniques such as evaporation or partial evaporation of the solvent, cooling, change of solvent, solvents or solvent mixtures, addition of crystallization seeds, etc. In a preferred embodiment the addition of cation A in step (b) of the process can induce crystallization or precipitation of the desired POM (Aₙ)^{m+}{[M₆(O₂)₉][(XM′₁₀O₃₇)₃]}^{m-}, wherein fractional crystallization is preferable. In a preferred embodiment, fractional crystallization might be accomplished by the slow addition of a specific amount of cation A to the reaction mixture of step (a) of the process or to its corresponding filtrates which might be beneficial for product purity and yield.

A preferred embodiment of the present invention is such a process wherein water is used as solvent and the at least one source of M is a water-soluble salt of Ir, Rh, Pt or Pd, preferably selected from K₂PtCl₄, PtCl₂, Pd(CH₃COO)₂, PdCl₂, Pd(NO₃)₂, PdSO₄, IrCl₃, or RhCl₃; and the at least one source of {X₈W₄₈O₁₈₄} is K₂₈Li₅H₇P₈W₄₈O₁₈₄.

A preferred embodiment of the present invention is such a process wherein the solvent contains water and the at least one source of {XM′₁₀O₃₇} is a water-soluble [XM′₉O₃₄]¹⁰⁻ salt of lithium, sodium, potassium, hydrogen or a combination thereof; more preferably a [GeW₉O₃₄]¹⁰⁻ and/or a [SiW₉O₃₄]¹⁰⁻ salt of sodium, lithium, potassium, hydrogen or a combination thereof; in particular Na₁₀[SiW₉O₃₄] and/or Na₁₀[GeW₉O₃₄].

A most preferred embodiment of the present invention is a process wherein in step (a) at least one source of M is used and wherein all M are the same; preferably wherein all M are Ce; more preferably wherein the solvent contains water and the at least one source of M is a water-soluble salt of Ce, in particular CeCl₃, Ce(NO₃)₃, Ce₂(SO₄)₃, or Ce(CH₃COO)₃. Another most preferred embodiment of the present invention is a process wherein in step (a) all M are Zr; more preferably wherein the solvent contains water and the at least one source of M is a water-soluble salt of Zr, such as ZrCl₄, Zr(NO₃)₄, Zr(SO₄)₂, or Zr(CH₃COO)₄, or all M are Hf, more preferably wherein the solvent contains water and the at least one source of M is a water-soluble salt of Hf, such as such as HfCl₄, Hf(NO₃)₄, Hf(SO₄)₂, or Hf(CH₃COO)₄.

According to one embodiment, the present POMs can be immobilized on a solid support. The present invention thus also relates to supported POMs comprising the POMs of the present invention or prepared by the process of the present invention on a solid support. Suitable supports include but are not limited to materials having a high surface area and/or a pore size which is sufficient to allow the POMs to be loaded, e.g., polymers, graphite, carbon nanotubes, electrode surfaces, aluminum oxide and aerogels of aluminum oxide and magnesium oxide, titanium oxide, zirconium oxide, cerium oxide, silicon dioxide, silicates, active carbon, mesoporous materials, like mesoporous silica, such as SBA-15 and MCM-41, zeolites, aluminophosphates (ALPOs), silicoaluminophosphates (SAPOs), metal organic frameworks (MOFs), zeolitic imidazolate frameworks (ZIFs), periodic mesoporous organosilicas (PMOs), and mixtures thereof and modified compounds thereof. Preferred supports are, for instance, mesoporous silica, more preferably SBA-15 or MCM-41, most preferably SBA-15. A variety of such solid supports is commercially available or can be prepared by common techniques. Furthermore, there are various common techniques to modify or functionalize solid supports, for example with regard to the size and shape of the surface or the atoms or groups available for bonding on the surface.

In a preferred embodiment of the present invention the immobilization of the POMs to the surface of the solid support is accomplished by means of adsorption, including physisorption and chemisorption, preferably physisorption. The adsorption is based on interactions between the POMs and the surface of the solid support such as van-der-Waals interactions, hydrogen-bonding interactions, ionic interactions, etc.

In a preferred embodiment the negatively charged polyanions {[M₆(O₂)₉][(XM′₁₀O₃₇)₃]} are adsorbed predominantly based on ionic interactions. Therefore, a solid support bearing positively charged groups is preferably used, in particular a solid support bearing groups that can be positively charged by protonation. A variety of such solid supports is commercially available or can be prepared by common techniques. In one embodiment the solid support is functionalized with positively charged groups, preferably groups that are positively charged by protonation, and the negatively charged polyanion {[M₆(O₂)₉][(XM′₁₀O₃₇)₃]} is linked to said positively charged groups by electrostatic interactions. In a preferred embodiment the solid support, preferably mesoporous silica, more preferably SBA-15 or MCM-41, most preferably SBA-15, is functionalized with moieties bearing positively charged groups, preferably tetrahydrocarbyl ammonium groups, more preferably groups that can be positively charged by protonation, most preferably mono-functionalized amino groups —NH₂. Preferably said groups are attached to the surface of the solid support by covalent bonds, preferably via a linker that comprises one or more, preferably one, of said groups, preferably an alkyl, aryl, alkenyl, alkynyl, hetero-alkyl, hetero-cycloalkyl, hetero-alkenyl, hetero-cycloalkenyl, hetero-alkynyl, hetero-aryl or cycloalkyl linker, more preferably an alkyl, aryl, hetero-alkyl or hetero-aryl linker, more preferably an alkyl linker, most preferably a methylene, ethylene, n-propylene, n-butylene, n-pentylene, n-hexylene linker, in particular a n-propylene linker. Preferably said linkers are bonded to any suitable functional group present on the surface of the solid support, such as to hydroxyl groups. Preferably said linkers are bonded to said functional groups present on the surface of the solid support either directly or via another group or atom, most preferably via another group or atom, preferably a silicon-based group, most preferably a silicon atom. In a most preferred embodiment of the present invention the POMs are supported on (3-aminopropyl)triethoxysilane (apts)-modified SBA-15.

In the supported POMs of the present invention, the POMs that are immobilized on the solid support are in the form of primary and/or secondary particles. In an especially preferred embodiment, the immobilized POMs particles are mainly in the form of primary particles (i.e. non-agglomerated primary particles), that is at least 90 wt % of the immobilized POMs particles are in the form of primary particles, preferably at least 95 wt %, more preferably at least 99 wt %, in particular substantially all the immobilized POMs particles are in the form of primary particles.

The invention is further directed to processes for preparing supported POMs according to the invention. Solid supports used in the context of this invention are as defined above. In a preferred embodiment of the present invention the surface of the solid supports is modified with positively charged groups, more preferably groups that can be positively charged by protonation. Those charged solid supports can be prepared by techniques well established in the art, for example by surface modification of a mesoporous silica, such as SBA-15, with a suitable reagent bearing a positively charged group or a group that can be positively charged by protonation, such as 3-aminopropyltriethoxysilane (apts), is conducted by heating, preferably under reflux, under inert gas atmosphere, such as argon or nitrogen, in an inert solvent with a suitable boiling point, such as hexane, heptane or toluene, for a suitable time, such as 4-8 hours, and finally the modified solid support is isolated, preferably by filtration, purified, preferably by washing, and dried, preferably under vacuum by heating, most preferably under vacuum by heating at about 100° C.

The optionally treated support may be further calcined at a temperature of 500° C. to 800° C. For the calcination, common equipment may be used, that is commercially available. Calcination of the optionally treated support may for instance be conducted under an oxygen containing gas such as air, under vacuum, under hydrogen or under an inert gas such as argon or nitrogen, more preferably under inert gas, most preferably under nitrogen.

The POMs according to the present invention or prepared by the process of the present invention can be immobilized on the surface of the solid support by contacting said POMs with the solid support. The present invention therefore also relates to a process for the preparation of supported POMs, comprising the step of contacting the POMs provided by the present invention or prepared according to the present invention with the solid support, thereby immobilizing at least part of the POMs onto the support; and optionally isolating the resulting supported POMs.

Said contacting may be conducted employing common techniques in the art, such as blending both the solid support and the POM in the solid form. In a preferred embodiment the POM is mixed with a suitable solvent, preferably water or an aqueous solvent, and the solid support is added to this mixture. In a more preferred embodiment the solid support is mixed with a suitable solvent, preferably water or an aqueous solvent, and the POM is added to this mixture. In case a solid support with groups that can be positively charged by protonation is used, the mixture is preferably acidified, for instance by addition of H₂SO₄, HNO₃ or HCl, most preferably by addition of H₂SO₄ or HNO₃, so that the pH value of the mixture ranges from 0.1 to 6, preferably from 1 to 4 and more preferably from 1.5 to 3, most preferably about 2. The mixture comprising POM, solid support and solvent is preferably stirred, typically for 1 min to 24 h, more preferably for 30 min to 15 h, more preferably for 1 h to 12 h, most preferably for 6 h to 10 h, in particular about 8 h. While stirring, the mixture may be at a temperature of from 20° C. to 100° C., preferably from 20° C. to 80° C., preferably from 20° C. to 60° C., preferably from 20° C. to 40° C., and most preferably about 25° C. Afterwards, the supported POM can be kept in the solvent as suspension or can be isolated. Isolation of the supported POM from the solvent may be performed by any suitable method in the art, such as by filtration, evaporation of the solvent, centrifugation or decantation, preferably by filtration or removal of the solvent in vacuum, more preferably by filtration. The isolated supported POMs may then be washed with a suitable solvent, preferably water or an aqueous solvent, and dried. Supported POMs may be dried in an oven at a temperature of e.g. about 100° C.

In supported POMs, the POM loading levels on the solid support may be up to 30 wt % or even more but are preferably up to 10 wt %, for instance up to 5 wt % or even up to 2 wt %. Accordingly, the POM loading level on the solid support is typically 0.01 to 30 wt %, particularly 0.05 to 20 wt %, more particularly 0.1 to 10 wt %, often 0.2-6 wt %, more often 0.3-5 wt %, and most often 0.5-2 wt %. POM loading levels on the solid support can be determined by elemental analysis such as Inductively Coupled Plasma Mass Spectrometry (ICP-MS) analysis, for instance using a Varian Vista MPX.

The invention is also directed to the use of optionally supported POMs provided by the present invention or prepared according to the present invention, for catalyzing homogeneous and heterogeneous oxidative conversion of organic substrates, such as oxidation of organic substrates.

In an especially preferred embodiment, said conversion of an organic substrate includes contacting the organic substrate with one or more optionally supported polyoxometalate in the optional presence of at least one source of oxygen, especially one source of oxygen, in particular at least one source of peroxo groups, more particularly one source of peroxo groups. Generally, in a preferred embodiment of the present invention, the at least one source of oxygen can be a gas containing dioxygen (O₂), e.g. air, ozone (O₃), nitrous oxide (N₂O), peroxodisulfate or a peroxide such as hydrogen peroxide (H₂O₂), an organic hydroperoxide (ROOH) e.g. tert-butylhydroperoxide, an organic dialkylperoxide (ROOR) e.g. tert-butyl peroxide, an organic diacyl peroxide (RC(O)OOC(O)R) e.g. diacetyl peroxide, a peroxy acid (RC(O)OOH) e.g. peracetic acid or m-chloroperbenzoic acid, or a peroxy ester (RC(O)OOR′). In an especially preferred embodiment, the source of oxygen is air, N₂O or a peroxide, in particular hydrogen peroxide.

In a first variant of this especially preferred embodiment, said contacting is conducted in the gas phase at a temperature of 200 to 500° C., preferably in the presence of air or N₂O as at least one source of oxygen. Contacting may take place at a pressure from atmospheric pressure to 100 atm.

In a second variant of this especially preferred embodiment, said contacting is conducted in the liquid phase, for instance at a temperature of 40 to 200° C., such as 45 to 90° C., under optional stirring, preferably under stirring.

Contacting may be conducted in the absence or presence of a solvent. In a first alternative of the present invention, contacting is conducted in the absence of a solvent, by contacting the neat organic substrate with one or more optionally supported polyoxometalate in the optional presence of at least one source of oxygen. In a second alternative of the present invention, contacting is conducted in the presence of a solvent such as water, acetonitrile, acetone, toluene, DMF, DMSO, ethanol, methanol, propanol, isopropanol, n-butanol, sec-butanol, isobutanol and mixtures thereof, for instance in an aqueous solution containing water and optional minor amounts of organic solvents.

In a further preferred embodiment of the present invention, contacting is conducted in the presence of a peroxide and the molar ratio of peroxide to organic substrate is in a range of from 0.01:1 to 50:1, preferably from 0.1:1 to 10:1, more preferably from 0.1:1 to 1:1.

The contacting of organic substrate with optionally supported POM and under the optional presence of at least one source of oxygen may, e.g., be carried out in a continuously stirred tank reactor (CSTR), a fixed bed reactor, a fluidized bed reactor or a moving bed reactor.

Another useful aspect of this invention is that the optionally supported POMs of the present invention can be recycled and used multiple times for the oxidative conversion of organic molecules, i.e., without significant loss.

In a preferred embodiment this invention thus also relates to a process comprising:
(a) contacting the first organic substrate with one or more optionally supported polyoxometalates under the optional presence of at least one source of oxygen,
(b) regenerating the spent polyoxometalates by adding at least one source of oxygen to the reaction mixture,
(c) contacting the one or more polyoxometalates with a sodium chloride aqueous solution at a temperature of 50° C. or more and recovering the one or more polyoxometalates by crystallization or precipitation, or filtering the one or more supported polyoxometalates and recovering the one or more supported polyoxometalates, to obtain recycled one or more optionally supported polyoxometalates;
(d) contacting the recycled one or more optionally supported polyoxometalates under optional addition of hydrogen peroxide with a second organic substrate which may be the same as or different from the first organic substrate; and
(e) optionally repeating steps (b) to (d).

The recycled optionally supported POMs of the present invention may be used on fresh organic molecules, or on recycled organic molecules from a recycle stream. Typically, the optionally supported POMs of the present invention can be recycled at least 1 time, preferably at least 4 times, preferably at least 8 times, preferably at least 12 times, preferably at least 100 times.

Due to the definite stoichiometry of POMs, the optionally supported POMs of the present invention can also be used as a precursor for mixed metal-oxide catalysts.

### EXAMPLES

The invention is further illustrated by the following examples.

### Example 1a: Synthesis of Lacunary Precursor Na₁₀[α-GeW₉O₃₄].25H₂O (“GeW₉”)

“GeW₉” was synthesized according to the procedure disclosed in G. Herve and A. Teze, Inorg. Chem., Vol. 16, No. 8, 2115-2117, 1977, incorporated herewith in its entirety. Sodium tungstate (182 g) and sodium germanate (11 g) were dissolved in water (200 mL). Hydrochloric acid (6 M, 130 mL) was added with stirring. The solution was then boiled for 1 hour and concentrated to 300 mL. Eventually, the germinate residue was filtered off. A solution of anhydrous sodium carbonate (50 g in 50 mL water) was added. The lukewarm solution was gently stirred and the sodium salt of the α-9-tungstogermanate precipitated. The FT-IR spectrum of “GeW₉” is shown in FIG. 1 (bottom curve). The number of attracted water molecules per polyanion was determined by Thermogravimetric analysis (TGA) as defined below.

### Example 1b: Synthesis of Na₂₄{[Ce₆(O₂)₉][(GeW₁₀O₃₇)₃]}.100H₂O

CeCl₃.7H₂O (0.064 g, 0.34 mmol) was dissolved in a 2 M sodium chloride solution (20 mL) at pH 5.0. Subsequently, “GeW₉” (0.5 g, 0.17 mmol), synthesized according to Example 1a was added while stirring. The reaction mixture was heated to 50° C. under stirring for 30 min. 30% hydrogen peroxide (1 mL, 1 mmol) was then poured into the solution while stirring and the solution was kept under stirring at 50° C. for another 1 h until the solution color turned to yellow. The reaction mixture was cooled to room temperature and filtered. The solution was left for one day in a closed vial. Then, 0.5 mL of 30% hydrogen peroxide were added dropwise and the mixture was heated under stirring to 50° C. for 15 min. The reaction mixture was then cooled to room temperature, filtered at room temperature and the resulting solution was left for crystallization in a closed vial. After one week, a dark red crystalline product was obtained. The product was collected by filtration and air-dried. Yield: 0.03 g. This product was analyzed by XRD, IR, elemental analysis, TGA and ¹⁸³W NMR and was identified as {[Ce₆(O₂)₉][(GeW₁₀O₃₇)₃]}²⁴⁻ polyanion (“Ce₆(GeW₁₀)₃”), isolated as hydrated salt Na₂₄{[Ce₆(O₂)₉][(GeW₁₀O₃₇)₃]}.100H₂O (“Na—Ce₆(GeW₁₀)₃”).

### Example 2: Analysis of “Na—Ce₆(GeW₁₀)₃”

The IR spectrum with 4 cm⁻¹ resolution was recorded on a Nicolet Avatar 370 FT-IR spectrophotometer on KBr pellet sample, from 400 to 1000 cm⁻¹. IR peaks are as follows (cm⁻¹): 3414, 2361, 2336, 1631, 1616, 1384, 940, 786, 667, 625, 620, 472, 453, 437. The FT-IR spectrum is shown in FIG. 1 (upper curve). The presence of peroxo ligands and terminal W═O groups in “Na—Ce₆(GeW₁₀)₃” is confirmed by the IR spectra. The appearance of the characteristic modes, at ca. 870, ca. 610, and ca. 530 cm⁻¹ indicates the presence of side-on peroxo linkage as n(O—O), v_{asym.}(W—O₂) and v_{sym.}(W—O), respectively. The spectral line near 964 cm⁻¹ n(W═O) proves the presence of terminal bonded W═O group.

Elemental analysis for “Na—Ce₆(GeW₁₀)₃” calculated (found): Na 5.03(5.02), Ce 7.65(7.76), Ge 1.96(1.97), W 50.03(50.1).

Thermogravimetric analysis (TGA) was performed on a SDT Q 600 device from TA Instruments with 10-30 mg samples in 100 μL alumina pans, under a 100 mL/min N₂ flow with a heating rate of 5° C./min between 0° C. and 800° C. (FIG. 2). The thermogram shows the step-wise loss of water molecules and peroxo groups as a function of temperature. The first weight loss occurs at 110° C., which corresponds to loss of water molecules from the outer sphere. However, the weight loss of 11.4% relates to the loss of coordinated water molecules. Furthermore, the peroxo groups are lost at 450-550° C.

### Example 3: Single Crystal X-Ray Diffraction (XRD) Data and Analysis of “Na—Ce₆(GeW₁₀)₃”

Besides IR, elemental analysis and TGA the product was also characterized by single-crystal XRD. The crystal was mounted on a Hampton cryoloop at 100 K using light oil for data collection. Indexing and data collection were carried on a Bruker Kappa X8 APEX II CCD single crystal diffractometer with a geometry and Mo Kα radiation (λ=0.71073 \acute{Å}). The SHELX software package (Bruker) was used to solve and refine the structure. An empirical absorption correction was applied using the SADABS program as disclosed in G. M. Sheldrick, SADABS, Program for empirical X-ray absorption correction, Bruker-Nonius: Madison, Wis. (1990). The structure was solved by direct method and refined by the full-matrix least squares method (Σw(|Fₒ|²−|F_{c}|²)²) with anisotropic thermal parameters for all heavy atoms included in the model. The H atoms were not located. Also, it was not possible to locate all lithium and potassium counter cations by XRD, due to crystallographic disorder. The exact number of counter cations and crystal water in the POM were thus based on elemental analysis and TGA. Compound “Na—Ce₆(GeW₁₀)₃” crystallizes in the triclinic space group P-1. Crystallographic data are detailed in Table 1.

TABLE 1
Crystal data for “Na—Ce₆(GeW₁₀)₃”

| | |
|---|---|
| Empirical formula | Na₂₄{[Ce^{IV}₆(O₂)₉][(GeW₁₀O₃₇)₃]}•100H₂O |
| Formula weight, g/mol | 9553.87 |
| Crystal system | Triclinic |
| Space group | P-1 |
| a, Å | 22.4086(9) |
| b, Å | 23.5105(8) |
| c, Å | 23.5550(11) |
| α, ° | 119.9095(17) |
| β, ° | 96.7633(31) |
| γ, ° | 103.2845(23) |
| Volume, Å³ | 10064.15(79) |
| Z | 2 |
| D_{calc}, g/cm³ | 3.153 |
| Absorption coefficient, | 18.951 |
| mm⁻¹ | |
| F(000) | 8296 |
| Crystal size, mm | 0.220 × 0.170 × 0.110 |
| Theta range for data | 1.760 to 25.000 |
| collection, ° | |
| Completeness to Θₘₐₓ % | |
| Limiting indices | −26 ≤ h ≤ 26, |
| | −27 ≤ k ≤ 27, |
| | −28 ≤ l ≤ 28 |
| Reflections collected/ | 404323/35369 |
| unique | |
| R(int) | 0.1303 |
| Refinement method | Full-matrix least-squares on F² |
| Goodness-of-fit on F² | 1.003 |
| Final R indices | R₁ = 0.0974, |
| | wR₂ = 0.2502 |
| R indices(all data) | R₁ = 0.1392, |
| | wR₂ = 0.3020 |

^{[a]} R₁ = Σ | |Fₒ| − |F_{c}| |/Σ |Fₒ|.
^{[b]} wR₂ = [Σw (Fₒ² − F_{c}²)²/Σw(Fₒ²)²]^{1/2}

### Example 4: Bond Valence Sum Values for Different Types of Oxygen Atoms in “Na—Ce₆(GeW₁₀)₃”

Bond valence sum calculations were determined ollowing the method disclosed in I. D. Brown and D. Altermant, Acta Crystallogr., Sect. B: Struct. Sci., 1985, 41, 244-247. These data are detailed in Table 2.

TABLE 2
Bond valence sum values for different types of
oxygen atoms in “Na—Ce₆(GeW₁₀)₃”

| Atom 1 | Atom 2 | d1, 2 [A⁰] | BVS value |
|---|---|---|---|
| Ce1 | | | |
| | O3C1 | 2.2336 | 0.574 |
| | O1H | 2.3204 | 0.454 |
| | O16H | 2.3366 | 0.442 |
| | O18H | 2.3404 | 0.430 |
| | O17H | 2.3429 | 0.431 |
| | O2H | 2.3561 | 0.418 |
| | O1C1 | 2.3956 | 0.370 |
| | O2C1 | 2.4487 | 0.321 |
| | O15C1 | 2.488 | 0.294 |
| | O3G1 | 2.833 | 0.144 |

### Example 5: Structure of the “Ce₆(GeW₁₀)₃” Polyanion

The structure of the “Ce₆(GeW₁₀)₃” polyanion and of the top and side views of the {Ce₆(O₂)₉}⁶⁺ core are displayed in FIGS. 3a and 3b. The three {GeW₁₀O₃₇}¹⁰⁻ units form a cyclic assembly encapsulating the {Ce₆(O₂)₉}⁶⁺ core. Furthermore, “Ce₆(GeW₁₀)₃” can be considered as a cyclic trimeric assembly containing three fused {[Ce^{IV}₂(O₂)₃][GeW₁₀O₃₇]}⁸⁻ monomers. The inner cyclic cerium-peroxo core consists of six Ce^{IV} centers, linked with each other by nine peroxo groups on both sides. The peroxo groups in each monomer show two different sets of connectivities in the structure. One of these is bridging two lacunary monomers containing cerium(IV) ions at the corners as (Ce⁴⁺—(O₂)₂—Ce⁴⁺) and the second is bridging two cerium ions within the same monomer as (Ce⁴⁺—(O₂)—Ce⁴⁺). The sodium cation in the center stabilizes the cyclic structure. All Ce⁴⁺ ions are also coordinated with trilacunary decatungstogermanate units by Ce—O(W) bonds.

### Example 6: ¹⁸³W NMR Spectrum of “Na—Ce₆(GeW₁₀)₃”

“Na—Ce₆(GeW₁₀)₃” crystals were dissolved in H₂O/D₂O (two drops of D₂O in 5 mL H₂O). ¹⁸³W NMR spectrum was recorded at room temperature on a 400 MHz JOEL ECX instrument. The chemical shift is reported with respect to the reference Na₂WO₄. The ¹⁸³W NMR spectrum is shown in FIG. 4.

“Na—Ce₆(GeW₁₀)₃” shows six peaks with an intensity ratio of 2:2:2:1:1, which correspond to the six types of non-equivalent W atoms according to the symmetry of “Ce₆(GeW₁₀)₃” as illustrated in FIG. 5. The four peaks down field with 2:2:2:2 intensity ratios are assigned to the four W-center pairs shown as striped polyhedrons in FIG. 5. The fifth peak at −230 ppm is assigned to the outer-triad tungsten (white polyhedron), and the up-field signal at about −300 ppm is assigned to the second triad tungsten (black polyhedron) which connects with the two Ce^{IV} centers, due to the significant difference in chemical environment.

### Example 7: Catalytic Oxidation of DL-Methionine Using “Ce₆(GeW₁₀)₃”

“Ce₆(GeW₁₀)₃” polyanion is stable in aqueous solution and was tested as a homogeneous catalyst for DL-methionine (S) oxidation to its sulfoxide (SO) and/or sulfone (SO₂) product(s). Conversion was followed in solution by ¹H NMR at room temperature using a 400 MHz JOEL ECX instrument. The chemical shift is reported with respect to the reference tetramethylsilane (TMS). ¹H NMR spectra of DL-methionine (S), its sulfoxide product (SO) and its sulfone product (SO₂) are shown in FIG. 6a.

In a first set of experiments, “Na—Ce₆(Ge₁₀)₃” (100 mg, 0.009 mmol) was mixed with DL-methionine (10 mg, 0.067 mmol) in aqueous solution (5 mL water) under micro-wave irradiation at 50° C. (power=30 W). The molar ratio of “Na—Ce₆(GeW₁₀)₃” to DL-methionine was 1:7.4. After 50 min, quantitative conversion of DL-methionine (S) to its sulfoxide product (SO) was observed but no conversion to the sulfone product (SO₂)(FIG. 6b, Curve (3)). Under similar conditions but at a molar ratio of “Na—Ce₆(GeW₁₀)₃” to DL-methionine 1:9, 90% conversion was observed. Similarly, if excess DL-methionine is added, it stays in the DL-methionine form without any oxidative conversion, indicating depletion of “Ce₆(GeW₁₀)₃” after conversion (FIG. 6b, Curve (4)).

Similar results were obtained by using hydrogen peroxide in the absence of “Ce₆(GeW₁₀)₃”: DL-methionine converted to its sulfoxide form but no further oxidation to the sulfone form was observed, even in the presence of excess hydrogen peroxide (FIG. 6b, Curve (1)). This was confirmed by the absence of reaction when methionine sulfoxide (SO) as a substrate is contacted with hydrogen peroxide: no formation of methionine sulfone (SO₂) was observed (FIG. 6b, Curve (2)).

In a second set of experiments, selective homogeneous oxidation of DL-methionine was performed using “Na—Ce₆(GeW₁₀)₃” (92 mg, 0.0084 mmol), DL-methionine (13 mg, 0.087 mmol) and 30% hydrogen peroxide (50 μl, 0.75 mmol) as a co-oxidant in aqueous solution (5 mL water). The reaction was performed under micro-wave irradiation at 50° C. (power=30 W) for 35 min. The results show that mixing “Na—Ce₆(GeW₁₀)₃” with DL-methionine in aqueous solution at a molar ratio of “Na—Ce₆(GeW₁₀)₃” to DL-methionine of 1:10 and in the presence of excess hydrogen peroxide leads to quantitative conversion of DL-methionine (S) to its sulfone product (SO₂) (FIG. 6b, Curve (5)). In particular, monitoring of the progress of the reaction in terms of product selectivity by ¹H NMR (FIG. 7) shows that all the DL-methionine (S) is transformed into its sulfoxide (SO) form within 1 min. With time, the sulfoxide form (SO) is oxidized further into the sulfone form (SO₂), reaching full conversion after 35 min. At room temperature, the product ratio after 25 min of reaction time of sulfoxide form (SO) to sulfone form (SO₂) is 80/20. Therefore, mild heating is needed to insure complete oxidation.

If more methionine was added to the reaction mixture, it also got converted, until all the hydrogen peroxide was exhausted, while further addition of 30% hydrogen peroxide (50 μl) to the reaction mixture regenerated the spent “Ce₆(GeW₁₀)₃” catalyst and restarted the catalytic process. The regeneration of the “Ce₆(GeW₁₀)₃” catalyst was recognizable by observing the solution color change from light yellow (sulfoxide formation pathway) or light red (sulfone formation pathway) to dark red. Said cycle was repeated ten times with the same catalytic turnover.

These results confirm the essential role of “Ce₆(GeW¹⁰)₃” to catalyze the sulfoxide (SO) to sulfone (SO₂) conversion as, in the presence of hydrogen peroxide but in the absence of “Ce₆(GeW₁₀)₃”, no formation of the sulfone form is observed.

The reaction pathways from DL-methionine (S) to its sulfoxide (SO) and/or sulfone (SO₂) product(s) are summarized in the scheme below.

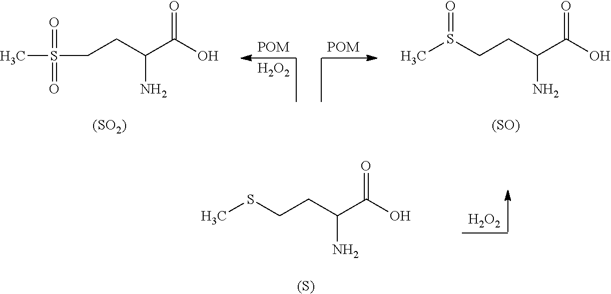

Quantitative catalyst recovery after several catalytic cycles was performed by further adding 2 M NaCl (2 mL) to the reaction mixture, in addition to 30% hydrogen peroxide (50 μl), the resulting turbid solution turning transparent upon heating at 50° C. for 5 min under stirring. Crystals of “Na—Ce₆(GeW₁₀)₃” were then collected by filtration from the clear solution after overnight cooling at room temperature. The integrity of the recrystallized “Na—Ce₆(GeW₁₀)₃” was confirmed by XRD and IR analysis.

### Example 8a: Synthesis of Precursor Na₁₀[α-SiW₉O₃₄].25H₂O (“SiW₉”)

“SiW₉” was synthesized according to the procedure disclosed in G. Herve and A. Teze, Inorg. Chem., Vol. 16, No. 8, 2115-2117, 1977, incorporated herewith in its entirety. Sodium tungstate (182 g) and sodium silicate (11 g) were dissolved in water (200 mL). Hydrochloric acid (6 M, 130 mL) was added with stirring. The solution was then boiled for 1 hour and concentrated to 300 mL. Eventually, the silicate residue was filtered off. A solution of anhydrous sodium carbonate (50 g in 50 mL water) was added. The lukewarm solution was gently stirred and the sodium salt of the α-9-tungstosilicate precipitated. The FT-IR spectrum of “SiW₉” is shown in FIG. 8 (bottom curve). The number of attracted water molecules per polyanion was determined by Thermogravimetric analysis (TGA) as defined below.

### Example 8b: Synthesis of Na₂₄{[Ce₆(O₂)₉][(SiW₁₀O₃₇)₃]}.100H₂O

Ce(NO₃)₃.6H₂O (0.074 g, 0.177 mmol) was dissolved in a 1 M sodium chloride solution (20 mL) at pH 5.0. Subsequently, “SiW₉” (0.5 g, 0.177 mmol), synthesized according to Example 8a was added while stirring. The reaction mixture was heated to 50° C. under stirring for 30 min. 30% hydrogen peroxide (1 mL, 1 mmol) was then poured into the solution while stirring and the solution was kept under stirring at 50° C. for another 30 min. The reaction mixture was cooled to room temperature and filtered. The solution was left for one week in a closed vial. Then, 0.5 mL of 30% hydrogen peroxide were added dropwise and the mixture was heated under stirring to 50° C. for 15 min. The reaction mixture was then left for crystallization in a closed vial. After one week, a dark red crystalline product was obtained. The product was collected by filtration and air-dried. Yield: 0.41 g. This product was analyzed by XRD, IR and TGA and was identified as {[Ce₆(O₂)₉][(SiW₁₀O₃₇)₃]}²⁴⁻ polyanion (“Ce₆(SiW₁₀)₃”), isolated as hydrated salt Na₂₄{[Ce₆(O₂)₉][(SiW₁₀O₃₇)₃]}.100H₂O (“Na—Ce₆(SiW₁₀)₃”).

### Example 9: Analysis of “Ce₆(SiW₁₀)₃”

The IR spectrum with 4 cm⁻¹ resolution was recorded on a Nicolet Avatar 370 FT-IR spectrophotometer on KBr pellet sample, from 400 to 1000 cm⁻¹. IR peaks are as follows (cm⁻¹): 1634, 988.54, 942.02, 885, 849, 776, 686, 667, 649, 525, 423, 420. The FT-IR spectrum is shown in FIG. 8. The presence of peroxo ligands and terminal W═O groups in “Na—Ce₆(SiW¹⁰)₃” is confirmed by the IR spectra. The appearance of the characteristic modes, at ca. 870, ca. 610, and ca. 530 cm⁻¹ indicates the presence of side-on peroxo linkage as n(O—O), v_{asym.}(W—O₂) and v_{sym.}(W—O), respectively. The spectral line near 964 cm⁻¹ n(W═O) proves the presence of terminal bonded W═O group.

Thermogravimetric analysis (TGA) was performed on a SDT Q 600 device from TA Instruments with 10-30 mg samples in 100 μL alumina pans, under a 100 mL/min N₂ flow with a heating rate of 5° C./min between 0° C. and 800° C. (FIG. 9). The thermogram shows the step-wise loss of water molecules and peroxo groups as a function of temperature. The first weight loss occurs at 110° C., which corresponds to loss of water molecules from the outer sphere. However, the weight loss of 14.2% at 275° C. relates to the loss of coordinated water molecules. Furthermore, the peroxo groups are lost at 450-550° C.

### Example 10: Single Crystal X-Ray Diffraction (XRD) Data and Analysis of “Na—Ce₆(SiW₁₀)₃”

Besides IR, elemental analysis and TGA the product was also characterized by single-crystal XRD. The crystal was mounted on a Hampton cryoloop at 100 K using light oil for data collection. Indexing and data collection were carried on a Bruker Kappa X8 APEX II CCD single crystal diffractometer with κ geometry and Mo Kα radiation (λ=0.71073 \acute{Å}). The SHELX software package (Bruker) was used to solve and refine the structure. An empirical absorption correction was applied using the SADABS program as disclosed in G. M. Sheldrick, SADABS, Program for empirical X-ray absorption correction, Bruker-Nonius: Madison, Wis. (1990). The structure was solved by direct method and refined by the full-matrix least squares method (Σw(|Fₒ|²−|F_{c}|²)²) with anisotropic thermal parameters for all heavy atoms included in the model. The H atoms were not located. Also, it was not possible to locate all lithium and potassium counter cations by XRD, due to crystallographic disorder. The exact number of counter cations and crystal water in the POM were thus based on elemental analysis and TGA. Compound “Na—Ce₆(SiW₁₀)₃” crystallizes in the triclinic space group P-1. Crystallographic data are detailed in Table 3.

TABLE 3
Crystal data for “Na—Ce₆(SiW₁₀)₃”

| | |
|---|---|
| Empirical formula | Na₂₄{[Ce^{IV}₆(O₂)₉][(SiW₁₀O₃₇)₃]}•100H₂O |
| Formula weight, g/mol | 10180.35 |
| Crystal system | Triclinic |
| Space group | P-1 |
| a, Å | 22.2638(7) |
| b, Å | 23.5142(7) |
| c, Å | 23.5205(6) |
| α, ° | 119.8558(13) |
| β, ° | 102.2457(17) |
| γ, ° | 99.0349(18) |
| Volume, Å³ | 9922.5(5) |
| Z | 2 |
| D_{calc}, g/cm³ | 3.407 |
| Absorption coefficient, | 18.836 |
| mm⁻¹ | |
| F(000) | 8948 |
| Crystal size, mm | 0.190 × 0.120 × 0.050 |
| Theta range for data | 1.380 to 28.435 |
| collection, ° | |
| Completeness to Θₘₐₓ % | |
| Limiting indices | −29 ≤ h ≤ 29, |
| | −31 ≤ k ≤ 31, |
| | −31 ≤ l ≤ 31 |
| Reflections collected/ | 476973/49714 |
| unique | |
| R(int) | 0.1131 |
| Refinement method | Full-matrix least-squares on F² |
| Goodness-of-fit on F² | 1.056 |
| Final R indices | R₁ = 0.0579, |
| | wR₂ = 0.1838 |
| R indices(all data) | R₁ = 0.0927, |
| | wR₂ = 0.2224 |

^{[a]} R¹ = Σ | |Fₒ| − |F_{C}| |/Σ |Fₒ|.
^{[b]} wR₂ = [Σw (Fₒ² − F_{c}²)²/Σw(Fₒ²)²]^{1/2}

### Example 11: Bond Valence Sum Values for Different Types of Oxygen Atoms in “Na—Ce₆(SiW₁₀)₃”

Bond valence sum calculations were determined ollowing the method disclosed in I. D. Brown and D. Altermatt, Acta Crystallogr., Sect. B: Struct. Sci., 1985, 41, 244-247. These data are detailed in Table 4.

TABLE 4
Bond valence sum values for different types of
oxygen atoms in “Na—Ce₆(SiW₁₀)₃”

| Atom 1 | Atom 2 | d1, 2 [A⁰] | BVS value |
|---|---|---|---|
| Ce1 | | | |
| | O3C1 | 2.2336 | 0.452 |
| | O1H | 2.3204 | 0.412 |
| | O16H | 2.3366 | 0.288 |
| | O18H | 2.3404 | 0.430 |
| | O17H | 2.3429 | 0.396 |
| | O2H | 2.3561 | 0.320 |
| | O1C1 | 2.3956 | 0.113 |
| | O2C1 | 2.4487 | 0.573 |
| | O15C1 | 2.488 | 0.433 |
| | O3G1 | 2.833 | 0.426 |

### Example 12: Structure of the “Ce₆(SiW₁₀)₃” Polyanion

The structure of the “Ce₆(SiW₁₀)₃” polyanion is displayed in FIG. 10. The three {SiW₁₀O₃₇}¹⁰⁻ units form a cyclic assembly encapsulating the {Ce₆(O₂)₉}⁶⁺ core. Furthermore, “Ce₆(SiW₁₀)₃” can be considered as a cyclic trimeric assembly containing three fused {[Ce^{IV}₂(O₂)₃][(SiW₁₀O₃₇)]}⁸⁻ monomers. The inner cyclic cerium-peroxo core consists of six Ce^{IV} centers, linked with each other by nine peroxo groups on both sides. The peroxo groups in each monomer show two different sets of connectivities in the structure. One of these is bridging two lacunary monomers containing cerium(IV) ions at the corners as (Ce⁴⁺—(O₂)₂—C⁴⁺) and the second is bridging two cerium ions within the same monomer as (Ce⁴⁺—(O₂)—Ce⁴⁺). The sodium cation in the center stabilizes the cyclic structure. All Ce⁴⁺ ions are also coordinated with trilacunary decatungstosilicate units by Ce—O(W) bonds.

All documents described herein are incorporated by reference herein, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including” for purposes of Australian law.

Additionally or alternately, the invention relates to:

### Embodiment 1

A polyoxometalate represented by the formula

(Aₙ)^{m+}{[M₆(O₂)₉][(XM′₁₀O₃₇)₃]}^{m-}

or solvates thereof, wherein
each A independently represents a cation,
n is the number of cations,
each M is independently selected from the group consisting of Ce, Ti, Zr, Hf, Nb, and Ta,
each X is independently selected from the group consisting of Ge, Si, P, and As,
each M′ is independently selected from the group consisting of W and Mo,
m is a number representing the total positive charge m+ of n cations A and the corresponding negative charge m− of the polyanion {[M₆(O₂)₉][(XM′₁₀O₃₇)₃]}.

Embodiment 2: Polyoxometalate according to embodiment 1, wherein all M are the same, preferably wherein all M are Ce, Zr or Hf, more preferably wherein all M are Ce^{IV}, Zr^{IV} or Hf^{IV}, most preferably wherein all M are Ce^{IV}.

Embodiment 3: Polyoxometalate according to embodiment 1 or 2, wherein all X are the same, preferably wherein all X are Ge or Si, more preferably all X are Ge^{IV} or Si^{IV}.

Embodiment 4: Polyoxometalate according to any one of the preceding embodiments, wherein all M′ are the same; preferably wherein all M′ are W^{VI} or Mo^{VI}, more preferably all M′ are W^{VI}.

Embodiment 5: Polyoxometalate according to any one of the preceding embodiments, wherein M is Ce, Zr or Hf, X is Ge or Si, and M′ is W or Mo.

Embodiment 6: Polyoxometalate according to any one of the preceding embodiments, wherein, each A is independently selected from the group consisting of Li, K, Na, Cs, ammonium, tetraalkyl ammonium, tetraalkyl phosphonium and combinations thereof, preferably from Li, K, Na, ammonium, tetraalkyl ammonium and combinations thereof and combinations thereof, more preferably wherein all A are the same, most preferably wherein all A are Na, Li or K.

Embodiment 7: Polyoxometalate according to any one of the preceding embodiments, represented by the formula

(Aₙ)^{m+}{[M₆(O₂)₉][(XM′₁₀O₃₇)₃]}^{m-}.wH₂O

wherein w represents the number of attracted water molecules per polyanion {[M₆(O₂)₉][(XM′₁₀O₃₇)₃]}, and ranges from 50 to 150, preferably from 70 to 130, more preferably from 80 to 120, most preferably from 90 to 110.

Embodiment 8: Polyoxometalate according to any one of the preceding embodiments, wherein the polyoxometalate is in the form of a solution-stable {[M₆(O₂)₉][(XM′₁₀O₃₇)₃]} polyanion, in particular in the form of an aqueous solution-stable polyanion.

Embodiment 9: Process for the preparation of the polyoxometalate of any one the preceding embodiments, said process comprising:
(a) reacting at least one source of M, at least one source of {XM′₁₀O₃₇}, and at least one source of oxygen to form a salt of the polyanion {[M₆(O₂)₉][(XM′₁₀O₃₇)₃]} or a solvate thereof,
(b) optionally adding at least one salt of A to the reaction mixture of step (a) to form a polyoxometalate (Aₙ)^{m+}{[M₆(O₂)₉][(XM′₁₀O₃₇)₃]}^{m-} or a solvate thereof, and
(c) recovering the polyoxometalate or solvate thereof.

Embodiment 10: Process according to embodiment 9, wherein the at least one source of {XM′₁₀O₃₇} is an XM′₉-based species, preferably an XM′₉O₃₄-based species, more preferably a [XM′₉O₃₄]¹⁰⁻ salt of lithium, sodium, potassium, hydrogen or a combination thereof.

Embodiment 11: Process according to embodiment 9 or 10, wherein in step (a) the concentration of the metal ions M originating from the source of M ranges from 0.001 to 1 mole/l, and the concentration of the heteroatoms X originating from the source of {XM′₁₀O₃₇} ranges from 0.0005 to 0.05 mole/l; preferably wherein the molar ratio M:X ranges from 1:1 to 3:1, for instance from 1.5:1 to 2.5:1 or from 1.8:1 to 2.2:1.

Embodiment 12: Process according to any one of embodiments 9 to 11, wherein in step (a) the source of oxygen is O₂, O₃ or N₂O and is added in excess of M or the source of oxygen is peroxodisulfate or a peroxide and is added in a molar ratio of peroxide to M of 3:1 to 10:1.

Embodiment 13: Process according to any one of embodiments 9 to 12, wherein water, an organic solvent or a combination thereof is used as solvent, preferably water or a combination of water with an organic solvent is used as solvent, in particular water is used as solvent.

Embodiment 14: Process according to any one of embodiments 9 to 13, wherein in step (a) at least one source of M is used and wherein all M are the same; preferably wherein all M are Ce, Zr or Hf; more preferably wherein the solvent contains water and the at least one source of M is a water-soluble salt of Ce, in particular CeCl₃, Ce(NO₃)₃, Ce₂(SO₄)₃, or Ce(CH₃COO)₃; or the at least one source of M is a water-soluble salt of Zr, in particular ZrCl₄, Zr(NO₃)₄, Zr(SO₄)₂, or Zr(CH₃COO)₄; or the at least one source of M is a water-soluble salt of Hf, in particular HfCl₄, Hf(NO₃)₄, Hf(SO₄)₂, or Hf(CH₃COO)₄.

Embodiment 15: Process according to any one of embodiments 9 to 14, wherein the solvent contains water and the at least one source of {XM′₁₀O₃₇} is a water-soluble [XM′₉O₃₄]¹⁰⁻ salt of lithium, sodium, potassium, hydrogen or a combination thereof; more preferably a [GeW₉O₃₄]¹⁰⁻ and/or a [SiW₉O₃₄]¹⁰⁻ salt of sodium, lithium, potassium, hydrogen or a combination thereof; in particular Na₁₀[SiW₉O₃₄] and/or Na₁₀[GeW₉O₃₄].

Embodiment 16: Process according to any one of embodiments 9 to 15, wherein step (a) is carried out in an aqueous solution, and the pH of the aqueous solution ranges from 1 to 10, preferably from 2 to 8, and more preferably from 3 to 7.

Embodiment 17: Process according to any one of embodiments 9 to 16, wherein at least one salt of A is added to the reaction mixture of step (a), such as NaCl, LiCl, KCl, NaBr, KBr, NaI, tetra-n-butylammonium chloride, or tetra-n-butylammonium bromide.

Embodiment 18: Process according to any one of embodiments 9 to 17, wherein in step (a) the reaction mixture is heated to a temperature of from 20° C. to 100° C., preferably from 40° C. to 80° C., more preferably from 45° C. to 60° C.

Embodiment 19: Supported polyoxometalate comprising polyoxometalate according to any one of embodiments 1 to 8 or prepared according to any one of embodiments 9 to 18, on a solid support.

Embodiment 20: Supported polyoxometalate according to embodiment 19, wherein the solid support is selected from polymers, graphite, carbon nanotubes, electrode surfaces, aluminium oxide and aerogels of aluminum oxide and magnesium oxide, titanium oxide, zirconium oxide, cerium oxide, silicon dioxide, silicates, active carbon, mesoporous silica, zeolites, aluminophosphates (ALPOs), silicoaluminophosphates (SAPOs), metal organic frameworks (MOFs), zeolitic imidazolate frameworks (ZIFs), periodic mesoporous organosilicas (PMOs), and mixtures thereof.

Embodiment 21: Process for the preparation of supported polyoxometalate according to embodiment 19 or 20, comprising the step of contacting polyoxometalate according to any one of embodiments 1 to 8 or prepared according to any one of embodiments 9 to 18, with a solid support.

Embodiment 22: Process for the homogeneous or heterogeneous oxidative conversion of organic substrate comprising contacting said organic substrate with the polyoxometalate of any one of embodiments 1 to 8 or prepared according to any one of embodiments 9 to 18 and/or with the supported polyoxometalate of embodiment 19 or 20 or prepared according to embodiment 21.

Embodiment 23: Process according to embodiment 22, wherein contacting the organic substrate with one or more optionally supported polyoxometalate is conducted in the gas phase at a temperature of 200 to 500° C., preferably in the presence of air or N₂O as at least one source of oxygen, preferably at a pressure from atmospheric pressure to 100 atm.

Embodiment 24: Process according to embodiment 22, wherein contacting the organic substrate with one or more optionally supported polyoxometalate is conducted in the liquid phase at a temperature of 40 to 200° C., preferably of 45 to 90° C., optionally in the presence of a solvent and/or of peroxodisulfate or a peroxide, preferably in the presence of peroxodisulfate or a peroxide in an aqueous solvent.

Embodiment 25: Process according to any one of embodiments 22 to 24, wherein peroxodisulfate or a peroxide is present and wherein the molar ratio of peroxodisulfate or peroxide to organic substrate is in a range of from 0.01:1 to 50:1, preferably from 0.1:1 to 10:1, more preferably from 0.1:1 to 1:1.

Embodiment 26: Process according to any one of embodiments 22 to 25, comprising:
(a) contacting the first organic substrate with one or more optionally supported polyoxometalates under the optional presence of at least one source of oxygen,
(b) regenerating the spent polyoxometalates by adding at least one source of oxygen to the reaction mixture,
(c) contacting the one or more polyoxometalates with a sodium chloride aqueous solution at a temperature of 50° C. or more and recovering the one or more polyoxometalates by crystallization or precipitation, or filtering the one or more supported polyoxometalates and recovering the one or more supported polyoxometalates, to obtain recycled one or more optionally supported polyoxometalates;
(d) contacting the recycled one or more optionally supported polyoxometalates under optional addition of at least one source of oxygen with a second organic substrate which may be the same as or different from the first organic substrate; and
(e) optionally repeating steps (b) to (d).

1. A polyoxometalate represented by the formula
(Aₙ)^{m+}{[M₆(O₂)₉][(XM′₁₀O₃₇)₃]}^{m-}
or solvates thereof, wherein
each A independently represents a cation,
n is the number of cations,
each M is independently selected from the group consisting of Ce, Ti, Zr, Hf, Nb, and Ta,
each X is independently selected from the group consisting of Ge, Si, P, and As,
each M′ is independently selected from the group consisting of W and Mo,
m is a number representing the total positive charge m+ of n cations A and the corresponding negative charge m− of the polyanion {[M₆(O₂)₉][(XM′₁₀O₃₇)₃]}.

2. Polyoxometalate according to claim 1, wherein all M are the same.

3. Polyoxometalate according to claim 1, wherein all X are the same.

4. Polyoxometalate according to claim 1, wherein all M′ are the same.

5. Polyoxometalate according to claim 1, wherein M is Ce, Zr or Hf, X is Ge or Si, and M′ is W or Mo.

6. Polyoxometalate according to claim 1, wherein, each A is independently selected from the group consisting of Li, K, Na, Cs, ammonium, tetraalkyl ammonium, tetraalkyl phosphonium and combinations thereof.

7. Polyoxometalate according to claim 1, represented by the formula
(Aₙ)^{m+}{[M₆(O₂)₉][(XM′₁₀O₃₇)₃]}^{m-}.wH₂O
wherein w represents the number of attracted water molecules per polyanion {[M₆(O₂)₉][(XM′₁₀O₃₇)₃]}, and ranges from 50 to 150.

8. Polyoxometalate according to claim 1, wherein the polyoxometalate is in the form of a solution-stable {[M₆(O₂)₉][(XM′₁₀O₃₇)₃]} polyanion, in particular in the form of an aqueous solution-stable polyanion.

9. Process for the preparation of the polyoxometalate of claim 1, said process comprising:
(a) reacting at least one source of M, at least one source of {XM′₁₀O₃₇}, and at least one source of oxygen to form a salt of the polyanion {[M₆(O₂)₉][(XM′₁₀O₃₇)₃]} or a solvate thereof,
(b) optionally adding at least one salt of A to the reaction mixture of step (a) to form a polyoxometalate (Aₙ)^{m+}{[M₆(O₂)₉][(XM′₁₀O₃₇)₃]}^{m-} or a solvate thereof, and
(c) recovering the polyoxometalate or solvate thereof.

10. Process according to claim 9, wherein the at least one source of {XM′₁₀O₃₇} is an XM′₉-based species.

11. Supported polyoxometalate comprising polyoxometalate according to claim 1, on a solid support.

12. Supported polyoxometalate according to claim 11, wherein the solid support is selected from polymers, graphite, carbon nanotubes, electrode surfaces, aluminium oxide and aerogels of aluminum oxide and magnesium oxide, titanium oxide, zirconium oxide, cerium oxide, silicon dioxide, silicates, active carbon, mesoporous silica, zeolites, aluminophosphates (ALPOs), silicoaluminophosphates (SAPOs), metal organic frameworks (MOFs), zeolitic imidazolate frameworks (ZIFs), periodic mesoporous organosilicas (PMOs), and mixtures thereof.

13. Process for the preparation of supported polyoxometalate, comprising the step of contacting the polyoxometalate according to claim 1 with a solid support.

14. Process for the homogeneous or heterogeneous oxidative conversion of organic substrate comprising contacting said organic substrate with the polyoxometalate of claim 1, optionally supported on a solid support.

15. Process according to claim 14, comprising:
(a) contacting a first organic substrate with one or more optionally supported polyoxometalates under the optional presence of at least one source of oxygen,
(b) regenerating the spent polyoxometalates by adding at least one source of oxygen to the reaction mixture,
(c) contacting the one or more polyoxometalates with a sodium chloride aqueous solution at a temperature of 50° C. or more and recovering the one or more polyoxometalates by crystallization or precipitation, or filtering the one or more supported polyoxometalates and recovering the one or more supported polyoxometalates, to obtain recycled one or more optionally supported polyoxometalates;
(d) contacting the recycled one or more optionally supported polyoxometalates under optional addition of a source of oxygen with a second organic substrate which may be the same as or different from the first organic substrate; and
(e) optionally repeating steps (b) to (d).

16. The polyoxometalate according to claim 1, wherein the M are Ce^{IV}, Zr^{IV} or Hf^{IV}, the X are Ge or Si, and the M′ are W^{VI} or Mo^{VI}.

17. The polyoxometalate according to claim 6, wherein each A is independently selected from the group consisting of Li, K, Na, ammonium, tetraalkyl ammonium and combinations thereof and combinations thereof.

18. The process according to claim 10, wherein the at least one source of {XM′₁₀O₃₇} is an a [XM′₉O₃₄]¹⁰⁻ salt of lithium, sodium, potassium, hydrogen or a combination thereof.
